(19) United States
(12) Patent Application Publication
(10) Pub. No.: US 2010/0317715 A1
(43) Pub. Date: Dec. 16, 2010

(54) METHODS FOR TREATING NEUROPSYCHIATRIC CONDITIONS

(76) Inventors: Benedikt Vollrath, San Diego, CA (US); Jay Lichter, Rancho Santa Fe, CA (US); Sergio G. Duron, San Diego, CA (US); Petpiboon Peppi Prasit, Rancho Santa Fe, CA (US)

Correspondence Address: WILSON, SONSINI, GOODRICH & ROSATI, 650 PAGE MILL ROAD, PALO ALTO, CA, 94304-1050, US

(21) Appl. No.: 12/745,886

(22) PCT Filed: Dec. 19, 2008
(86) PCT No.: PCT/US2008/087837

§ 371 Date: Aug. 30, 2010

Related U.S. Application Data
(60) Provisional application No. 61/016,315, filed on Dec. 21, 2007.
(60) Provisional application No. 61/022,760, filed on Jan. 22, 2008.
(60) Provisional application No. 61/035,314, filed on Mar. 10, 2008.

(51) Int. Cl. A61K 31/713; A61P 25/00

(52) U.S. Cl. 514/44 A

(57) ABSTRACT

Provided herein are compositions and methods for treating a subject suffering from Fragile X syndrome, autism, Down's syndrome, mental retardation, or a neuropsychiatric condition (e.g., schizophrenia). The methods include systemic administration of a a therapeutically effective amount of a PAK inhibitor in combination with a Group I mGluR antagonist (e.g., an mGluR5 antagonist). The PAK inhibitor and mGluR antagonist can be administered together, e.g., in one pharmacological composition, or they can be administered separately.

### CROSS REFERENCE

This application claims the benefit of U.S. provisional application Ser. No. 61/035,314 filed Mar. 10, 2008, U.S. provisional application Ser. No. 61/022,760 filed Jan. 22, 2008, and U.S. provisional application Ser. No. 61/016,315 filed Dec. 21, 2007, each of which is incorporated by reference in its entirety.

### BACKGROUND OF THE INVENTION

Neuropsychiatric conditions (NCs) are characterized by a variety of debilitating affective and cognitive impairments. For example, in schizophrenia, one of the most common psychotic disorders, individuals may suffer from hallucinations, disorders of movement, and the inability to initiate plans, speak, or express emotion. Cognitive deficits in schizophrenia include problems with attention, memory, and the executive functions that allow us to plan and organize. Other NCs include, e.g., mood disorders, age-related cognitive decline, and neurological disorders (e.g., epilepsy and Huntington's disease). The effects of NCs are devastating to the quality of life of those afflicted as well as that of their families. Moreover, NCs impose an enormous health care burden on society. A number of NCs, as well as other conditions that affect cognitive function, have been associated with alterations in the morphology and/or density of dendritic spines, membranous protrusions from dendritic shafts of neurons that serve as highly specialized structures for the formation, maintenance, and function of synapses.

### SUMMARY OF THE INVENTION

Described herein are methods and compositions for treating a subject suffering from a neuropsychiatric condition (e.g., schizophrenia, clinical depression, age-related cognitive decline, Huntington's disease, and epilepsy) by administering to the subject a pharmaceutical composition comprising a therapeutically effective amount of an inhibitor of a p21-activated kinase (PAK), e.g., PAK1, PAK2 or PAK3, as described herein. PAK activation is shown to play a key role in spine morphogenesis, and inhibitors of PAK are administered to rescue defects in spine morphogenesis in subjects suffering from a condition in which dendritic spine morphology, density, and/or function are aberrant, including but not limited to abnormal spine density, spine size, spine morphology, spine plasticity, spine motility. In addition, metabotropic glutamate receptors (mGluRs) are involved in the regulation of spine morphogenesis.

In one aspect provided herein is a method for treating a subject suffering from a neuropsychiatric condition, comprising administering to the subject a pharmacological composition comprising a therapeutically effective amount of at least one inhibitor of a p21activated kinase (PAK), wherein the neuropsychiatric disorder is associated with abnormal spine density or abnormal spine morphology. In some embodiments, the at least one inhibitor is an inhibitor of a Group I PAK, such as, for example, PAK1, PAK2, and/or PAK3.

In some embodiments, administering a pharmacological composition comprising a therapeutically effective amount of one or more of a PAK transcription inhibitor, a PAK clearance agent, an agent that binds PAK to prevent its interaction with one or more cellular or extracellular proteins, or a PAK antagonist to a subject alleviates one or more symptoms or pathologies of the disorder. In some embodiments, administering a pharmacological composition comprising a therapeutically effective amount of one or more of a PAK transcription inhibitor, a PAK clearance agent, an agent that binds PAK to prevent its interaction with one or more cellular proteins, or a PAK antagonist to a subject delays the onset of one or more symptoms or pathologies of a neuropsychiatric disorder.

In one aspect provided herein is a method for delaying loss of dendritic spine density in a subject with a neuropsychiatric condition or predicted to develop a neuropsychiatric condition, comprising administering to the subject a pharmacological composition comprising a therapeutically effective amount of at least one of the compounds chosen from the group consisting of: a PAK transcription inhibitor, a PAK clearance agent, an agent that binds PAK to prevent its interaction with one or more natural binding partners, and a PAK antagonist. In some preened embodiments, the pharmacological composition comprises a therapeutically effective amount of at least one of the compounds chosen from the group consisting of: a Group I PAK transcription inhibitor, a Group I PAK clearance agent, an agent that binds a Group I PAK to prevent its interaction with one or more cellular proteins, and a Group I PAK antagonist. The subject is in some preferred embodiments a human.

In one aspect provided herein is a method for increasing dendritic spine density in a subject with a neuropsychiatric condition, comprising administering to the subject a pharmacological composition comprising a therapeutically effective amount of at least one of the compounds chosen from the group consisting of: a PAK transcription inhibitor, a PAK clearance agent, an agent that binds PAK to prevent its interaction with one or more natural binding partners, and a PAK antagonist. In some preferred embodiments, the pharmacological composition comprises a therapeutically effective amount of at least one of the compounds chosen from the group consisting of: a Group I PAK transcription inhibitor, a Group I PAK clearance agent, an agent that binds a Group I PAK to prevent its interaction with one or more cellular proteins, and a Group I PAK antagonist. The subject is in some preferred embodiments a human.

In one aspect provided herein is a method for reversing some or all defects in dendritic spine morphology, spine size and/or spine plasticity in a subject with a neuropsychiatric condition or predicted to develop a neuropsychiatric condition, comprising administering to the subject a pharmacological composition comprising a therapeutically effective amount of at least one of the compounds chosen from the group consisting of: a PAK transcription inhibitor, a PAK clearance agent, an agent that binds PAK to prevent its interaction with one or more natural binding partners, and a PAK antagonist. In some preferred embodiments, the pharmacological composition comprises a therapeutically effective amount of at least one of the compounds chosen from the group consisting of: a Group I PAK transcription inhibitor, a Group I PAK clearance agent, an agent that binds a Group I PAK to prevent its interaction with one or more cellular proteins, and a Group I PAK antagonist. The subject is in some preferred embodiments a human.

In some embodiments, the neuropsychiatric disorder is schizophrenia, depression, epilepsy, age-related cognitive decline, Huntington's disease, or Parkinson's disease.

In some embodiments, where the neuropsychiatric disorder is schizophrenia, the method further comprises administering to the subject a therapeutically effective amount of an antipsychotic drug. In some embodiments, where the neuropsychiatric condition is depression, the method further comprises administering to the subject a therapeutically effective amount of an antidepressant drug.

In some embodiments, the at least one inhibitor of a p21activated kinase comprises a small molecule inhibitor. In some embodiments, the small molecule inhibitor is EKB-569 or a derivative thereof. In some embodiments, the small molecule inhibitor is TKI-258 or a derivative thereof. In some embodiments, the small molecule inhibitor is SU-14813 or a derivative thereof.

In some embodiments, the at least one inhibitor of a p21activated kinase comprises FMRP or a fragment thereof that interacts with the p21-activated kinase. In some embodiments, the at least one inhibitor comprises a polypeptide comprising the KH domains of FMRP.

In some embodiments, the at least one inhibitor of a p21activated kinase comprises a fragment of the huntingtin protein that interacts with the p21-activated kinase. In some embodiments, the at least one inhibitor comprises a compound that binds to the p21-activated kinase at a site on the p21-activated kinase that interacts with the mutant form of huntingtin protein.

In some embodiments, the at least one inhibitor inhibits expression of the p21-activated kinase. In some embodiments, that at least one inhibitor that inhibits expression of the p21-activated kinase comprises a PAK RNAi nucleic acid, a PAK antisense nucleic acid, a PAK ribozyme, or any combination thereof.

In some embodiments, the at least one inhibitor of a p21-activated kinase comprises a PAK autoinhibitory domain polypeptide or a fragment thereof.

In some embodiments, a p21-activated kinase to be inhibited comprises one or more of a Group I PAK, for example, PAK1, PAK2, and/or PAK3. In some embodiments, a p21-activated kinase to be inhibited comprises PAK1. In some embodiments, a p21-activated kinase to be inhibited comprises PAK2. In some embodiments, a p21-activated kinase to be inhibited comprises PAK3. In some embodiments, a p21-activated kinase to be inhibited comprises PAK-4. In some embodiments, a p21-activated kinase to be inhibited comprises PAK5. In some embodiments, a p21-activated kinase to be inhibited comprises PAK6.

In another aspect provided herein is a method for treating a subject suffering from schizophrenia, comprising administering to the subject a pharmacological composition comprising a therapeutically effective amount of an inhibitor of a p21-activated kinase.

In a further aspect provided herein is a method for treating a subject suffering from a mood disorder, comprising administering to the subject a pharmacological composition comprising a therapeutically effective amount of an inhibitor of a p21-activated kinase.

In a related aspect provided herein is a method for treating a subject suffering from depression, comprising administering to the subject a pharmacological composition comprising a therapeutically effective amount of an inhibitor of a p21-activated kinase.

In yet another aspect provided herein is a method for treating a subject suffering from age-related cognitive decline, comprising administering to the subject a pharmacological composition comprising a therapeutically effective amount of an inhibitor of a p21-activated kinase.

In a further aspect provided herein is a method for treating a subject suffering from Alzheimer's disease, comprising administering to the subject a pharmacological composition comprising a therapeutically effective amount of an inhibitor of a p21-activated kinase.

In a further aspect provided herein is a method for treating a subject suffering from Huntington's disease, comprising administering to the subject a pharmacological composition comprising a therapeutically effective amount of an inhibitor of a p21-activated kinase.

Accordingly, in one aspect provided herein is a pharmacological composition comprising at least one inhibitor of a p21-activated kinase and at least one antagonist of a Group I mGluR. In some embodiments, the at least one inhibitor comprises a small molecule inhibitor of a p21-activated kinase. In some embodiments, the small molecule inhibitor is EKB569 or a derivative thereof. In some embodiments, the small molecule inhibitor is TKI-258 or a derivative thereof. In some embodiments, the small molecule inhibitor is SU-14813 or a derivative thereof.

In some embodiments, the Group I mGluR antagonist is an mGluR5-selective antagonist. In some embodiments, the mGluR5-selective antagonist is selected from the Group 1 mGluR antagonists presented in U.S. Pat. No. 7,205,441; U.S. Pat. No. 6,482,824; or U.S. Patent Application Publication No. 2007/0208028. In some embodiments, the Group I mGluR antagonist is (E)-6-methyl-2-styryl-pyridine (SIB 1893), 6-methyl-2-(phenylazo)-3-pyridinol, .alpha.-methyl-4-carboxyphenylglycine (MCPG), or 2-methyl-6-(phenylethynyl)-pyridine (MPEP).

In another aspect provided herein is a method for treating a subject suffering from Fragile X syndrome, autism, mental retardation, or Down's Syndrome, comprising administering to the subject a therapeutically effective amount of at least one inhibitor of a p21-activated kinase and a therapeutically effective amount of at least one Group I mGluR antagonist. In some embodiments, at least one inhibitor to be administered inhibits expression of the p21activated kinase. In some embodiments, at least one inhibitor to be administered includes a small molecule inhibitor.

In some embodiments, the mGluR5-selective antagonist to be administered is selected from the Group 1 mGluR antagonists presented in U.S. Pat. No. 7,205,441; U.S. Pat. No. 6,482,824; or U.S. Patent Application Publication No. 2007/0208028. In some embodiments, the Group I mGluR antagonist to be administered is (E)-6-methyl-2-styryl-pyridine (SIB 1893), 6-methyl-2-(phenylazo)-3-pyridinol, .alpha.-methyl-4-carboxyphenylglycine (MCPG), or 2-methyl-6-(phenylethynyl)-pyridine (MPEP).

In some embodiments, the at least one inhibitor and the at least one antagonist are administered separately to the subject. In some embodiments, the at least one inhibitor and the at least one antagonist are administered by different routes of administration. In some embodiments, the subject is administered a pharmacological composition comprising the at least one inhibitor of p21-activated kinase and the at least one antagonist of a Group I mGluR.

In some embodiments, the Group I mGluR antagonist is administered at a dose ranging from about 0.01 to about 5 mg/kg body weight/day.

In some embodiments, the subject to be treated is suffering from Fragile X syndrome. In some embodiments, the subject to be treated is suffering from autism. In some embodiments, the subject to be treated is suffering from Down's syndrome. In some embodiments, the subject to be treated is suffering from Huntington's disease. In some embodiments, the subject to be treated has a mutation in the hit gene that is diagnostic of the development of Huntington's disease. In a further aspect provided herein is a method for treating a subject suffering from Fragile X syndrome, comprising administering to the subject a therapeutically effective amount of at least one inhibitor of a p21-activated kinase and a therapeutically effective amount of at least one Group I mGluR antagonist.

In another aspect provided herein is a method for treating a subject suffering from schizophrenia, comprising administering to the subject a therapeutically effective amount of at least one inhibitor of a p21-activated kinase and a therapeutically effective amount of at least one Group I mGluR antagonist.

In another aspect provided herein is a method for treating a subject suffering from a neuropsychiatric condition, comprising administering to the subject a therapeutically effective amount of at least one inhibitor of a p21activated kinase and a therapeutically effective amount of at least one Group I mGluR antagonist.

### CERTAIN DEFINITIONS

As used herein the term “Treatment” or “treating” includes achieving a therapeutic benefit and/or a prophylactic benefit. By therapeutic benefit is meant eradication or amelioration of the underlying disorder or condition being treated. For example, in an individual with schizophrenia, therapeutic benefit includes partial or complete halting of the progression of the disorder, or partial or complete reversal of the disorder. Also, a therapeutic benefit is achieved with the eradication or amelioration of one or more of the physiological or psychological symptoms associated with the underlying condition such that an improvement is observed in the patient, notwithstanding the fact that the patient is still affected by the condition. A prophylactic benefit of treatment includes prevention of a condition, retarding the progress of a condition, or decreasing the likelihood of occurrence of a condition. As used herein, “treating” or “treatment” includes prophylaxis.

As used herein, the phrase “neuropsychiatric condition” refers to any condition, other than Fragile-X Mental Retardation, that results in chronic impairment in cognition, affect, or motor function.

As used herein, the phrase “psychotic disorder” refers to a severe mental disorder characterized by derangement of personality and loss of contact with reality and causing deterioration of normal social functioning. Examples of psychotic disorders include, but are not limited to, schizophrenia, schizoaffective disorder, schizophreniform disorder, brief psychotic disorder, delusional disorder, shared psychotic disorder (Folie a Deux), substance induced psychosis, and psychosis due to a general medical condition.

As used herein, the phrase “cognitive disorder” refers to any chronic condition that impairs reasoning ability, e.g., Alzheimer's Disease, Huntington's disease, age-related cognitive decline, or Pick's disease.

As used herein, the phrase “abnormal spine size” refers to dendritic spine volumes or dendritic spine surface areas associated with a neuropsychiatric condition that deviate significantly relative to spine volumes or surface areas in the same brain region (e.g., the CA1 region) in a normal subject (e.g., a mouse, rat, or human) of the same age. The phrase “defective spine morphology” or “abnormal spine morphology” refer to abnormal dendritic spine shapes associated with a neuropsychiatric condition relative to the dendritic spine shapes observed in the same region in a normal subject (e.g., a mouse, rat, or human) of the same age. The phrase “abnormal spine plasticity” refers to a defect of dendritic spines to undergo stimulus-dependent morphological or functional synaptic or post-synaptic changes (e.g., calcium entry through NMDA receptors, LTP, LTD, etc) associated with a neuropsychiatric condition as compared to dendritic spines in the same brain region in a normal subject of the same age. The “defect” in dendritic spine plasticity includes, e.g., a reduction in dendritic spine plasticity or an excess level of dendritic plasticity (e.g., a lower threshold for induction of LTP, LTD, spine remodeling etc.). The phrase “abnormal spine motility” refers to an excessively low or high movement of dendritic spines in response to synaptic or pharmacological stimuli (e.g., actin-based movement) associated with a neuropsychiatric condition as compared to dendritic spines in the same brain region in a normal subject of the same age.

As used herein, the term “inhibitor” refers to a molecule which is capable of inhibiting one or more of the biological activities of a target molecule, such as a TrkB receptor, an Eph receptor, an NMDA receptor, GRB2, NCK, CDK5, rac, Cdc42, NCK, ETK, PDK1, a PI3 kinase or the like. Inhibitors, for example, act by reducing or suppressing the activity of a target molecule and/or reducing or suppressing signal transduction. In some embodiments, the phrase “partial inhibitor” refers to a molecule which can induce a partial response. In some instances, a partial inhibitor mimics the spatial arrangement, electronic properties, or some other physicochemical and/or biological property of the inhibitor. In some instances, in the presence of elevated levels of an inhibitor, a partial inhibitor competes with the inhibitor for occupancy of the target molecule and provides a reduction in efficacy, relative to the inhibitor alone. In some embodiments, a PAK inhibitor described herein is a partial inhibitor of a PAK.

As used herein, the phrase “biologically active” refers to a characteristic of any substance that has activity in a biological system and/or organism. For instance, a substance that, when administered to an organism, has a biological effect on that organism, is considered to be biologically active. In particular embodiments, where a protein or polypeptide is biologically active, a portion of that protein or polypeptide that shares at least one biological activity of the protein or polypeptide is typically referred to as a “biologically active” portion.

As used herein, the term “effective amount” is an amount, which when administered systemically, is sufficient to effect beneficial or desired results, such as beneficial or desired clinical results, or enhanced cognition, memory, mood, or other desired effects. An effective amount is also an amount that produces a prophylactic effect, e.g., an amount that delays, reduces, or eliminates the appearance of a pathological or undesired condition. Such conditions include, but are not limited to, schizophrenia, clinical depression, epilepsy, age-related cognitive decline, Huntington's disease, Down's syndrome, Niemann-Pick disease, spongiform encephalitis, Lafora disease, Maple syrup urine disease, maternal phenylketonuria, atypical phenylketonuria, or tuberous sclerosis. An effective amount is optionally administered in one or more administrations. In terms of treatment, an “effective amount” of a composition described herein is an amount that is sufficient to palliate, ameliorate, stabilize, reverse or slow the progression of an NC, e.g., age-related cognitive decline. An “effective amount” includes any PAK inhibitor used alone or in conjunction with one or more agents used to treat a disease or disorder. An “effective amount” of a therapeutic agent as described herein will be determined by a patient's attending physician or other medical care provider. Factors which influence what a therapeutically effective amount will be include, the absorption profile (e.g., its rate of uptake into the brain) of a PAK inhibitor, time elapsed since the initiation of the NC, and the age, physical condition, existence of other disease states, and nutritional status of the individual being treated. Additionally, other medication the patient is receiving, e.g., antipsychotic drugs used in combination with a PAK inhibitor, will typically affect the determination of the therapeutically effective amount of the therapeutic agent to be administered.

As used herein, the phrase “an agent that facilitates the transport of the PAK inhibitor across the blood brain barrier” refers to an agent that mediates, facilitates and/or enhances penetration of a compound described herein through the blood brain barrier. In some embodiments, a blood brain barrier facilitator increases influx of a compound described herein. In some instances, an increase in influx of a compound described herein across the blood brain bather is achieved by modulating the lipophilic nature of a compound described herein (e.g, via conjugation of a low density lipid particle to a compound described herein). In some instances, an increase in influx of a compound described herein across the blood brain bather is achieved by modifying a compound described herein (e.g., by reducing or increasing the number of charged groups on the compound) and enhancing affinity for a blood brain barrier transporter. In some embodiments, a blood brain barrier facilitator reduces or inhibits the efflux of a compound described herein from the blood brain barrier (e.g., an agent that suppresses P-glycoprotein pump mediated efflux).

As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; (4) post-translational modification of a polypeptide or protein.

As used herein the term “PAK polypeptide” or “PAK protein” refers to a protein that belongs in the family of p21-activated serine/threonine protein kinases. These include mammalian isoforms of PAK, e.g., the Group I PAK proteins (sometimes referred to as Group A PAK proteins), including PAK1, PAK2, PAK3, as well as the Group II PAK proteins (sometimes referred to as Group B PAK proteins), including PAK-4, PAK5, and/or PAK6 Also included as PAK polypeptides or PAK proteins are lower eukaryotic isoforms, such as the yeast Ste20 (Leberter at al., 1992, EMBO J., 11:4805; incorporated herein by reference) and/or the Dictyostelium single-headed myosin I heavy chain kinases (Wu et al., 1996, J. Biol. Chem., 271:31787; incorporated herein by reference). Representative examples of PAK include, but are not limited to, human PAK1 (GenBank Accession Number AAA65441), human PAK2 (GenBank Accession Number AAA65442), human PAK3 (GenBank Accession Number AAC36097), human PAK 4 (GenBank Accession Numbers NP_{—}005875 and CAA09820), human PAK5 (GenBank Accession Numbers CAC18720 and BAA94194), human PAK6 (GenBank Accession Numbers NP_{—}064553 and AAF82800), human PAK7 (GenBank Accession Number Q9P286), C. elegans PAK (GenBank Accession Number BAA11844), D. melanogaster PAK (GenBank Accession Number AAC47094), and rat PAK1 (GenBank Accession Number AAB95646). In some embodiments, a PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, AAC36097, NP_{—}005875, CAA09820, CAC18720, BAA94194, NP_{—}064553, AAF82800, Q9P286, BAA11844, AAC47094, and/or AAB95646. In some embodiments, a Group I PAK polypeptide comprises an amino acid sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers AAA65441, AAA65442, and/or AAC36097.

Representative examples of PAK genes encoding PAK proteins include, but are not limited to, human PAK1 (GenBank Accession Number U24152), human PAK2 (GenBank Accession Number U24153), human PAK3 (GenBank Accession Number AF068864), human PAK4 (GenBank Accession Number AJ011855), human PAK5 (GenBank Accession Number AB040812), and human PAK6 (GenBank Accession Number AF276893). In some embodiments, a PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, AF068864, AJ011855, AB040812, and/or AF276893. In some embodiments, a Group I PAK gene comprises a nucleotide sequence that is at least 70% to 100% identical, e.g., at least 75%, 80%, 85%, 86%, 87%, 88%, 90%, 91%, 92%, 94%, 95%, 96%, 97%, 98%, or any other percent from about 70% to about 100% identical to sequences of GenBank Accession Numbers U24152, U24153, and/or AF068864.

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent homology between the two sequences is a function of the number of identical positions shared by the sequences (i.e., % identity=# of identical positions/total # of positions (e.g., overlapping positions)×100). In one embodiment the two sequences are the same length.

To determine percent homology between two sequences, the algorithm of Karlin and Altschul (1990) Proc. Natl. Acad. Sci. USA 87:2264-2268, modified as in Karlin and Altschul (1993) Proc. Natl. Acad. Sci. USA 90:5873-5877 is used. Such an algorithm is incorporated into the NBLAST and XBLAST programs of Altschul, et al. (1990) J. Mol. Biol. 215:403-410. BLAST nucleotide searches are performed with the NBLAST program, score=100, wordlength=12 to obtain nucleotide sequences homologous to a nucleic acid molecules described or disclose herein. BLAST protein searches are performed with the XBLAST program, score=50, wordlength=3. To obtain gapped alignments for comparison purposes, Gapped BLAST is utilized as described in Altschul et al. (1997) Nucleic Acids Res. 25:3389-3402. When utilizing BLAST and Gapped BLAST programs, the default parameters of the respective programs (e.g., XBLAST and NBLAST) are used. See the website of the National Center for Biotechnology Information for further details (on the world wide web at ncbi.nlm.nih.gov). Proteins suitable for use in the methods described herein also includes proteins having between 1 to 15 amino acid changes, e.g., 1, 2, 3, 4, 5, 6, 7, 8, 9, 10, 11, 12, 13, 14, or 15 amino acid substitutions, deletions, or additions, compared to the amino acid sequence of any protein PAK inhibitor described herein. In other embodiments, the altered amino acid sequence is at least 75% identical, e.g., 77%, 80%, 82%, 85%, 88%, 90%, 92%, 95%, 97%, 98%, 99%, or 100% identical to the amino acid sequence of any protein PAK inhibitor described herein. Such sequence-variant proteins are suitable for the methods described herein as long as the altered amino acid sequence retains sufficient biological activity to be functional in the compositions and methods described herein. Where amino acid substitutions are made, the substitutions should be conservative amino acid substitutions. Among the common amino acids, for example, a “conservative amino acid substitution” is illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff et al (1992), Proc. Natl. Acad. Sci. USA, 89:10915-10919). Accordingly, the BLOSUM62 substitution frequencies are used to define conservative amino acid substitutions that may be introduced into the amino acid sequences described or disclosed herein. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

As used herein, the term “PAK activity,” unless otherwise specified, includes, but is not limited to, at least one of PAK protein-protein interactions, PAK phosphotransferase activity (intermolecular or intermolecular), translocation, etc of one or more PAK isoforms.

As used herein, a “PAK inhibitor” refers to any molecule, compound, or composition that directly or indirectly decreases the PAK activity. In some embodiments, PAK inhibitors inhibit, decrease, and/or abolish the level of a PAK mRNA and/or protein or the half-life of PAK mRNA and/or protein, such inhibitors are referred to as “clearance agents”. In some embodiments, a PAK inhibitor is a PAK antagonist that inhibits, decreases, and/or abolishes an activity of PAK. In some embodiments, a PAK inhibitor also disrupts, inhibits, or abolishes the interaction between PAK and its natural binding partners (e.g., a substrate for a PAK kinase, a Rac protein, a cdc42 protein, LIM kinase) or a protein that is a binding partner of PAK in a pathological condition, as measured using standard methods.

In some embodiments, PAK inhibitors reduce, abolish, and/or remove the binding between PAK and at least one of its natural binding partners (e.g., Cdc42 or Rac). Thus, binding between PAK and at least one of its natural binding partners is stronger in the absence of the inhibitor than in its presence. In some embodiments, PAK inhibitors prevent, reduce, or abolish binding between PAK and a protein that abnormally accumulates or aggregates in cells or tissue a disease state, such as for example, alpha synuclein in Parkinson's disease, beta amyloid or tau in Alzheimer's disease, FMRP in Fragile-X Mental Retardation, or the huntingtin protein in Huntington's disease. Thus, binding between PAK and at least one of the proteins that aggregates or accumulates in a neuropsychiatric disorder or cognitive disorder is stronger in the absence of the inhibitor than in its presence.

Alternatively or additionally, PAK inhibitors inhibit the phosphotransferase activity of PAK, e.g., by binding directly to the catalytic site or by altering the conformation of PAK such that the catalytic site becomes inaccessible to substrates. In some embodiments, PAK inhibitors inhibit the ability of PAK to phosphorylate at least one of its target substrates, e.g., LIM kinase 1 (LIMK1), myosin light chain kinase (MLCK); or itself, i.e., autophosphorylation. PAK inhibitors include inorganic and/or organic compounds.

A “subject” or an “individual,” as used herein, is an animal, for example, a human patient. In some embodiments a “subject” or an “individual” is a human. In some embodiments, the subject suffers from schizophrenia, clinical depression, epilepsy, or age-related cognitive decline.

In some embodiments, a pharmacological composition comprising a PAK inhibitor is “administered peripherally” or “peripherally administered.” As used herein, these terms refer to any form of administration of an agent, e.g., a therapeutic agent, to an individual that is not direct administration to the CNS, i.e., that brings the agent in contact with the non-brain side of the blood-brain barrier. “Peripheral administration,” as used herein, includes intravenous, intra-arterial, subcutaneous, intramuscular, intraperitoneal, transdermal, by inhalation, transbuccal, intranasal, rectal, oral, parenteral, sublingual, or trans-nasal. In some embodiments, a PAK inhibitor is administered by an intracerebral route.

The terms “polypeptide,” and “protein” are used interchangeably herein to refer to a polymer of amino acid residues. That is, a description directed to a polypeptide applies equally to a description of a protein, and vice versa. The terms apply to naturally occurring amino acid polymers as well as amino acid polymers in which one or more amino acid residues is a non-naturally occurring amino acid, e.g., an amino acid analog. As used herein, the terms encompass amino acid chains of any length, including full length proteins (i.e., antigens), wherein the amino acid residues are linked by covalent peptide bonds.

The term “amino acid” refers to naturally occurring and non-naturally occurring amino acids, as well as amino acid analogs and amino acid mimetics that function in a manner similar to the naturally occurring amino acids. Naturally encoded amino acids are the 20 common amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, and valine) and pyrolysine and selenocysteine. Amino acid analogs refers to compounds that have the same basic chemical structure as a naturally occurring amino acid, i.e., an α carbon that is bound to a hydrogen, a carboxyl group, an amino group, and an R group, such as, homoserine, norleucine, methionine sulfoxide, methionine methyl sulfonium. Such analogs have modified R groups (such as, norleucine) or modified peptide backbones, but retain the same basic chemical structure as a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “nucleic acid” refers to deoxyribonucleotides, deoxyribonucleosides, ribonucleosides, or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless specifically limited otherwise, the term also refers to oligonucleotide analogs including PNA (peptidonucleic acid), analogs of DNA used in antisense technology (phosphorothioates, phosphoroamidates, and the like). Unless otherwise indicated, a particular nucleic acid sequence also implicitly encompasses conservatively modified variants thereof (including but not limited to, degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)).

The terms “isolated” and “purified” refer to a material that is substantially or essentially removed from or concentrated in its natural environment. For example, an isolated nucleic acid is one that is separated from the nucleic acids that normally flank it or other nucleic acids or components (proteins, lipids, etc. . . . ) in a sample. In another example, a polypeptide is purified if it is substantially removed from or concentrated in its natural environment. Methods for purification and isolation of nucleic acids and proteins are documented methodologies.

The term “antibody” describes an immunoglobulin whether natural or partly or wholly synthetically produced. The term also covers any polypeptide or protein having a binding domain which is, or is homologous to, an antigen-binding domain. CDR grafted antibodies are also contemplated by this term.

The term antibody as used herein will also be understood to mean one or more fragments of an antibody that retain the ability to specifically bind to an antigen, (see generally, Holliger et al., Nature Biotech. 23 (9) 1126-1129 (2005)). Non-limiting examples of such antibodies include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544 546), which consists of a VII domain; and (vi) an isolated complementarity determining region (CDR). Furthermore, although the two domains of the Fv fragment, VL and VH, are coded for by separate genes, they are optionally joined, using recombinant methods, by a synthetic linker that enables them to be made as a single protein chain in which the VL and VH regions pair to form monovalent molecules (known as single chain Fv (scFv); see e.g., Bird et al. (1988) Science 242:423 426; and Huston et al. (1988) Proc. Natl. Acad. Sci. USA 85:5879 5883; and Osbourn et al. (1998) Nat. Biotechnol. 16:778). Such single chain antibodies are also intended to be encompassed within the term antibody. Any VH and VL sequences of specific scFv is optionally linked to human immunoglobulin constant region cDNA or genomic sequences, in order to generate expression vectors encoding complete IgG molecules or other isotypes. VH and VL are also optionally used in the generation of Fab, Fv or other fragments of immunoglobulins using either protein chemistry or recombinant DNA technology. Other forms of single chain antibodies, such as diabodies are also encompassed.

“F(ab′)₂” and “Fab”′ moieties are optionally produced by treating immunoglobulin (monoclonal antibody) with a protease such as pepsin and papain, and includes an antibody fragment generated by digesting immunoglobulin near the disulfide bonds existing between the hinge regions in each of the two H chains. For example, papain cleaves IgG upstream of the disulfide bonds existing between the hinge regions in each of the two H chains to generate two homologous antibody fragments in which an L chain composed of VL (L chain variable region) and CL (L chain constant region), and an H chain fragment composed of VH (H chain variable region) and CHγ1 (γ1 region in the constant region of H chain) are connected at their C terminal regions through a disulfide bond. Each of these two homologous antibody fragments is called Fab′. Pepsin also cleaves IgG downstream of the disulfide bonds existing between the hinge regions in each of the two H chains to generate an antibody fragment slightly larger than the fragment in which the two above-mentioned Fab′ are connected at the hinge region. This antibody fragment is called F(ab′)2.

The Fab fragment also contains the constant domain of the light chain and the first constant domain (CH1) of the heavy chain. Fab′ fragments differ from Fab fragments by the addition of a few residues at the carboxyl terminus of the heavy chain CH1 domain including one or more cysteine(s) from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are documented.

“Fv” is the minimum antibody fragment which contains a complete antigen-recognition and antigen-binding site. This region consists of a dimer of one heavy chain and one light chain variable domain in tight, non-covalent association. It is in this configuration that the three hypervariable regions of each variable domain interact to define an antigen-binding site on the surface of the VH-VL dimer. Collectively, the six hypervariable regions confer antigen-binding specificity to the antibody. However, even a single variable domain (or half of an Fv comprising only three hypervariable regions specific for an antigen) has the ability to recognize and bind antigen, although at a lower affinity than the entire binding site.

“Single-chain Fv” or “sFv” antibody fragments comprise a VH, a VL, or both a VH and VL domain of an antibody, wherein both domains are present in a single polypeptide chain. In some embodiments, the Fv polypeptide further comprises a polypeptide linker between the VH and VL domains which enables the sFv to form the desired structure for antigen binding. For a review of sFv see, e.g., Pluckthun in The Pharmacology of Monoclonal Antibodies, Vol. 113, Rosenburg and Moore eds. Springer-Verlag, New York, pp. 269 315 (1994).

A “chimeric” antibody includes an antibody derived from a combination of different mammals. The mammal is, for example, a rabbit, a mouse, a rat, a goat, or a human. The combination of different mammals includes combinations of fragments from human and mouse sources.

In some embodiments, an antibody described or disclosed herein is a monoclonal antibody (MAb), typically a chimeric human-mouse antibody derived by humanization of a mouse monoclonal antibody. Such antibodies are obtained from, e.g., transgenic mice that have been “engineered” to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. In some embodiments, the transgenic mice synthesize human antibodies specific for human antigens, and the mice are used to produce human antibody-secreting hybridomas.

### INCORPORATION BY REFERENCE

All publications, patents, and patent applications mentioned in this specification are herein incorporated by reference for the subject matter on which they are cited.

### DETAILED DESCRIPTION OF FIGURES

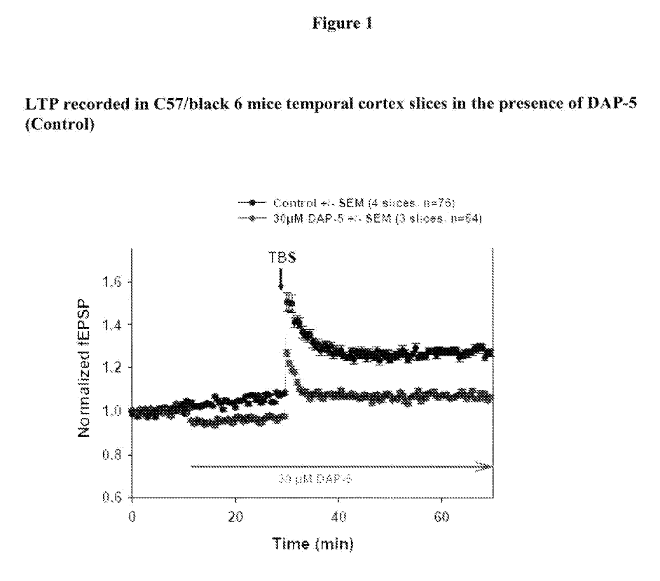

FIG. 1. LTP recorded in C57/black 6 mice temporal cortex slices in the presence of 30 μM D-AP5 (Control)

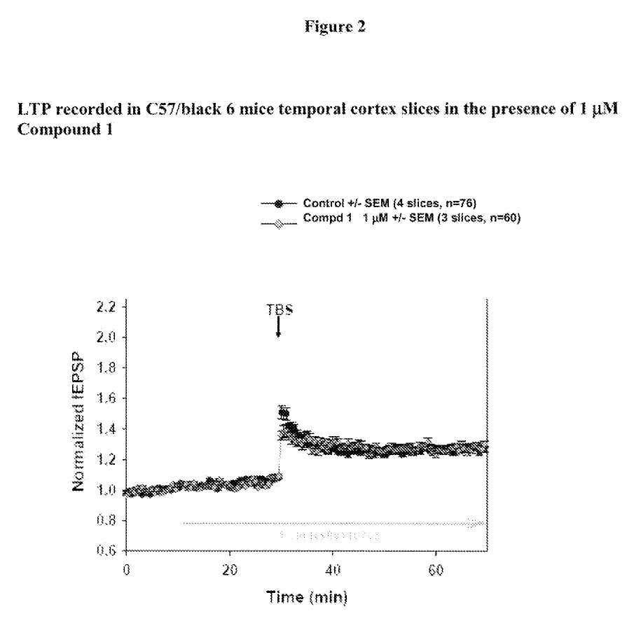

FIG. 2. LTP recorded in C57/black 6 mice temporal cortex slices in the presence of 1 μM Compound 1.

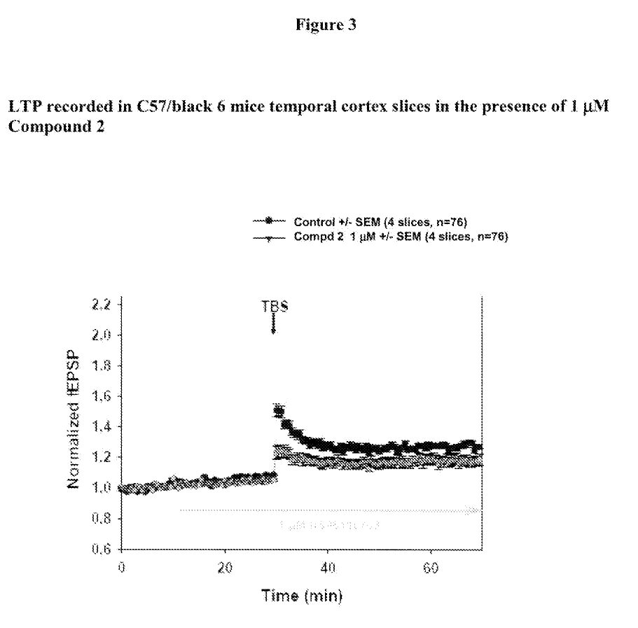

FIG. 3. LTP recorded in C57/black 6 mice temporal cortex slices in the presence of 1 μM Compound 2.

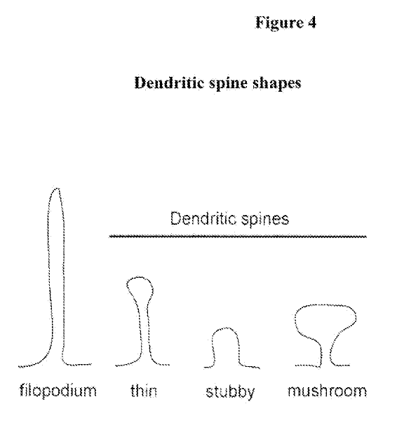

FIG. 4. Dendritic spine shapes

### DETAILED DESCRIPTION OF THE INVENTION

A number of NCs are characterized by abnormal dendritic spine morphology, spine size, spine plasticity and/or spine density as described in a number of studies referred to herein. On the other hand PAK kinase activity has been implicated in spine morphogenesis, maturation, and maintenance. See, e.g., Kreis et al (2007), J Biol Chem, 282(29):21497-21506; Hayashi et al (2007), Proc Natl Acad Sci USA., 104(27):11489-11494, Hayashi et al (2004), Neuron, 42(5):773-787; Penzes et al (2003), Neuron, 37:263-274. Thus, in the methods for treating NCs described herein PAK activity is inhibited by administering a PAK inhibitor to rescue defects in spine morphology, size, plasticity and/or density associated with NCs as described herein. NCs that are treated by the methods described herein include, but are not limited to, pscyhotic disorders, mood disorders, age-related cognitive decline, epilepsy, Huntington's disease, Down's syndrome, Niemann-Pick disease, spongiform encephalitis, Lafora disease, Maple syrup urine disease, maternal phenylketonuria, atypical phenylketonuria, and tuberous sclerosis. In some embodiments, one or more PAK inhibitors are used in combination with one or more Group I metabotropic glutamate receptor (mGluR) antagonists (e.g., mGluR5 antagonists) to treat a subject suffering from Fragile X syndrome, Down's syndrome, autism, mental retardation, or schizophrenia. The combination of PAK inhibitors with Group I mGluR antagonists allows a reduced dose of both agents to be used thereby reducing the likelihood of side effects associated with higher dose monotherapies. In one embodiment, the Group I mGluR antagonist dose is reduced in the combination therapy by at least 50% relative to the corresponding monotherapy dose, whereas a PAK inhibitor dose is not reduced relative to the monotherapy dose; in further embodiments, the reduction in Group I mGluR antagonist dose is at least 75%; in yet a further embodiment, the reduction in Group I mGluR antagonist dose is at lest 90%. Symptoms and diagnostic criteria for all of the conditions mentioned above are described in detail in the Diagnostic and Statistical Manual of Mental Disorders, fourth edition, American Psychiatric Association (2005) (DSM-IV).

Abnormal dendritic spine morphology and/or density have been found in a number of NCs as described below. Accordingly, in some embodiments, the methods described herein are used to treat a subject suffering from a neuropsychiatric condition that is associated with abnormal dendritic spine density, spine size, spine plasticity, spine morphology, spine plasticity, or spine motility. In some embodiments, the methods described herein are used to treat a subject suffering from a psychotic disorder. Examples of psychotic disorders include, but are not limited to, schizophrenia, schizoaffective disorder, schizophreniform disorder, brief psychotic disorder, delusional disorder, shared psychotic disorder (Folie a Deux), substance induced psychosis, and psychosis due to a general medical condition. See, e.g., Black et al. (2004), Am Psychiatry, 161:742-744; Broadbelt et al. (2002), Schizophr Res, 58:75-81; Glantz et al. (2000), Arch Gen Psychiatry 57:65-73; and Kalus et al. (2000), Neuroreport, 11:3621-3625.

In some embodiments, the methods described herein are used to treat a subject suffering from a mood disorder. Examples of mood disorders include, but are not limited to, clinical depression, bipolar disorder, cyclothymia, and dysthymia. See, e.g., Hajszan et al (2005), Eur J Neurosci, 21:1299-1303; Law et al (2004) Am J Psychiatry, 161(10):1848-1855; Norrholm et al. (2001), Synapse, 42:151-163; and Rosoklija et al. (2000), Arch Gen Psychiatry, 57:349-356.

In some embodiments, the methods described herein are used to treat a subject suffering from age-related cognitive decline. See, e.g., Dickstein et al (2007), Aging Cell, 6:275-284; and Page et al. (2002), Neuroscience Letters, 317:37-41.

In some embodiments, the methods described herein are used to treat a subject suffering from epilepsy. See, e.g., Wong (2005), Epilepsy and Behavior, 7:569-577; Swann et al (2000), Hippocampus, 10:617-625; and Jiang et al (1998), J Neurosci, 18(20):8356-8368.

In some embodiments, the methods described herein are used to treat a subject suffering from Parkinson's Disease or Huntington's Disease. See, e.g., Neely et al (2007), Neuroscience, 149(2):457-464; Spires et al (2004), Eur J Neurosci, 19:2799-2807; Klapstein et al (2001), J Neurophysiol, 86:2667-2677; Ferrante et al (1991), J Neurosci, 11:3877-3887; and Graveland et al (1985), Science, 227:770-773.

In some embodiments, the methods described herein are used to treat a subject suffering from Down's syndrome, Niemann-Pick disease, spongiform encephalitis, Lafora disease, Maple syrup urine disease, maternal phenylketonuria, atypical phenylketonuria, and tuberous sclerosis.

In some embodiments, a composition containing a therapeutically effective amount of a PAK inhibitor is administered prophylactically to a subject that while not overtly manifesting symptoms of a NC has been identified as having a high risk of developing a NC, e.g., the subject is identified as being a carrier of a mutation or polymorphism associated with Huntington's disease (The Huntington's Disease Collaborative Research Group (1993) Cell 72: 971-83) clinical depression (see, e.g., Hashimoto at al (2006), Hum Mol Genet, 15(20):3024-3033) or schizophrenia (see, e.g., Hall et al (2006), Nat Neurosci., 9(12):1477-8), or the subject is from a family that has a high incidence of a particular NC. In some embodiments, MRI is used to detect brain morphological changes in children prior to the onset of schizophrenia (see, e.g., Toga at al (2006), TINS, 29(3):148-159). For some NCs, risk is age-dependent. For example, the typical age of onset for schizophrenia is between 20-28 for males and 26-32 for females. Accordingly, in some embodiments, a PAK inhibitor is administered to a subject at risk between about 1 to about 10 years, e.g., 1, 2, 3, 4, 5, 6, 7, 8, 9, or 10 years prior to an established age range of onset for a particular NC.

p21-Activated Kinases (PAKs)

The PAKs constitute a family of serine-threonine kinases that is composed of “conventional”, or Group I PAKs, that includes PAK1, PAK2, and PAK3, and “non-conventional”, or Group H PAKs, that includes PAK-4, PAK5, and PAK6. See, e.g., Zhao et al. (2005), Biochem J, 386:201-214. These kinases function downstream of the small GTPases Rac and/or Cdc42 to regulate multiple cellular functions, including dendritic morphogenesis and maintenance (see, e.g., Ethell et al (2005), Prog in Neurobiol, 75:161-205; Penzes et al (2003), Neuron, 37:263-274), motility, morphogenesis, angiogenesis, and apoptosis, (see, e.g., Bokoch et al., 2003, Annu. Rev. Biochem., 72:743; and Hofmann et al., 2004, J. Cell Sci., 117:4343;). GTP-bound Rac and/or Cdc42 bind to inactive PAK, releasing steric constraints imposed by a PAK autoinhibitory domain and/or permitting PAK auto-phosphorylation and/or activation. Numerous autophosphorylation sites have been identified that serve as markers for activated PAK.

Prominent upstream effectors of PAK include, but are not limited to, TrkB receptors; NMDA receptors; EphB receptors; FMRP; Rho-family GTPases, including Cdc42, Rac (including but not limited to Rac1 and Rac2), Chp, TC10.

In some instances, downstream effectors of PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47phox, BAD, caspase 3, estrogen and/or progesterone receptors, RhoGEF, GEF-H1, NET1, Gαz, phosphoglycerate mutase-B, RhoGDI, prolactin, p41Arc, and/or Aurora-A (See, e.g., Bokoch at al., 2003, Annu. Rev. Biochem., 72:743; and Hofmann at al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Paxillin; NESH; SH3-containing proteins (e.g. Nck and/or Grb2); kinases (e.g. Akt, PDK1, PI 3-kinase/p85, Cdk5, Cdc2, Src kinases, Ab1, and/or protein kinase A (PKA)); and/or phosphatases (e.g. phosphatase PP2A, POPX1, and/or POPX2).

Prominent downstream targets of mammalian PAK include, but are not limited to, substrates of PAK kinase, such as Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47phox, BAD, caspase 3, estrogen and/or progesterone receptors, RhoGEF, GEF-H1, NET1, Gαz, phosphoglycerate mutase-B, RhoGDI, prolactin, p41Arc, and/or Aurora-A (See, e.g., Bokoch et al., 2003, Annu. Rev. Biochem., 72:743; and Hofmann et al., 2004, J. Cell Sci., 117:4343). Other substances that bind to PAK in cells include CIB; sphingolipids; lysophosphatidic acid, G-protein β and/or γ subunits; PIX/COOL; GIT/PKL; Nef; Paxillin; NESH; SH3-containing proteins (e.g. Nck and/or Grb2); kinases (e.g. Akt, PDK1, PI 3-kinase/p85, Cdk5, Cdc2, Src kinases, Ab1, and/or protein kinase A (PKA)); and/or phosphatases (e.g. phosphatase PP2A, POPX1, and/or POPX2).

### PAK Inhibitors

As described herein, a subject suffering from a NC is treated by administration of a pharmaceutical composition containing a PAK inhibitor. In some embodiments, a PAK inhibitor is a Group I PAK inhibitor that inhibits, for example, one or more Group I PAK polypeptides, for example, PAK1, PAK2, and/or PAK3. In some embodiments, a PAK inhibitor is a PAK1 inhibitor. In some embodiments, a PAK inhibitor is a PAK2 inhibitor. In some embodiments, a PAK inhibitor is a PAK3 inhibitor. In some embodiments, a PAK inhibitor is a Group II PAK inhibitor that inhibits one or more Group II PAK polypeptides that inhibits, for example PAK-4, PAK5, and/or PAK6. In some embodiments, a PAK inhibitor is a PAK-4 inhibitor. In some embodiments, a PAK inhibitor is a PAK-4 inhibitor. In some embodiments, a PAK inhibitor is a PAK5 inhibitor. In some embodiments, a PAK inhibitor is a PAK6 inhibitor.

In some embodiments, a PAK inhibitor is a compound of Formula I:

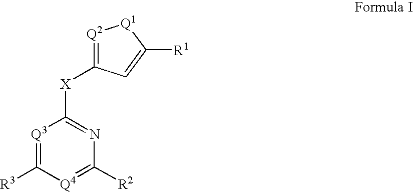

wherein:

R¹, R² and R³ are independently H, halo, hydroxy, cyano, substituted or unsubstituted alkyl, substituted or unsubstituted alkoxy, substituted or unsubstituted alkylamine, substituted or unsubstituted heteroallyl, substituted or unsubstituted aryl, substituted or unsubstituted heteroaryl, substituted or unsubstituted cycloalkyl, or substituted or unsubstituted heterocycloalkyl;

Q¹, Q², Q³ and Q⁴ are independently N, N—R^{4a} or C—R^{4b}; wherein R^{4a} is H, substituted or unsubstituted alkyl; R^{4b} is H, halo, hydroxy, cyano, substituted or unsubstituted alkyl; or substituted or unsubstituted alkoxy;
X is O, N—R⁵ or C(R⁵)₂, wherein each R⁵ is independently H, hydroxy, substituted or unsubstituted alkyl; or two R⁵ taken together are (═O) or (═NR^{5a}); wherein R^{5a} is H, hydroxy, substituted or unsubstituted alkyl, or substituted or unsubstituted alkoxy;
   or a pharmaceutically acceptable salt thereof.

In some embodiments, X is N—R⁵. In some embodiments, X is NH. In some embodiments, Q¹ is N, NH or CH. In some embodiments, Q² is N, NH or CH. In some embodiments, Q¹ is NH and Q² is N. In some embodiments, Q³ is N or CH. In some embodiments, Q⁴ is N or CH. In some embodiments, Q³ and Q⁴ are N.

In some embodiments, R¹ is substituted or unsubstituted aryl, substituted or unsubstituted heteroaryl, substituted or unsubstituted cycloalkyl, or substituted or unsubstituted heterocycloalkyl. In some embodiments, R¹ is substituted or unsubstituted aryl, substituted or unsubstituted heteroaryl. In some embodiments, R¹ is substituted or unsubstituted cycloalkyl, or substituted or unsubstituted heterocycloalkyl. In some embodiments, R¹ is substituted or unsubstituted cyclopropane, substituted or unsubstituted cyclobutane, substituted or unsubstituted cyclopentane, substituted or unsubstituted cyclohexane, substituted or unsubstituted pyran, substituted or unsubstituted tetrahydrofuran, substituted or unsubstituted aziridine, substituted or unsubstituted azetidine, substituted or unsubstituted oxetane, substituted or unsubstituted azetidinone, substituted or unsubstituted oxetone, substituted or unsubstituted pyrrolidine, substituted or unsubstituted piperidine, substituted or unsubstituted pyrrolidinone or substituted or unsubstituted piperidinone.

In some embodiments, R² is substituted or unsubstituted aryl, substituted or unsubstituted heteroaryl, substituted or unsubstituted cycloalkyl, or substituted or unsubstituted heterocycloalkyl. In some embodiments, R² is substituted or unsubstituted aryl, substituted or unsubstituted heteroaryl. In some embodiments, R² is substituted or unsubstituted cycloalkyl, or substituted or unsubstituted heterocycloalkyl. In some embodiments, R² is substituted or unsubstituted cyclopropane, substituted or unsubstituted cyclobutane, substituted or unsubstituted cyclopentane, substituted or unsubstituted cyclohexane, substituted or unsubstituted pyran, substituted or unsubstituted tetrahydrofuran, substituted or unsubstituted aziridine, substituted or unsubstituted azetidine, substituted or unsubstituted oxetane, substituted or unsubstituted azetidinone, substituted or unsubstituted oxetone, substituted or unsubstituted pyrrolidine, substituted or unsubstituted piperidine, substituted or unsubstituted pyrrolidinone or substituted or unsubstituted piperidinone.

In some embodiments, R³ is substituted or unsubstituted alkyl, substituted or unsubstituted alkoxy, or substituted or unsubstituted alkylamine. In some embodiments, R³ is substituted or unsubstituted alkylaryl, substituted or unsubstituted alkylheteroaryl, substituted or unsubstituted alklylcycloalkyl, or substituted or unsubstituted alkylheterocycloalkyl.

In some embodiments, a compound of Formula I has the structure:

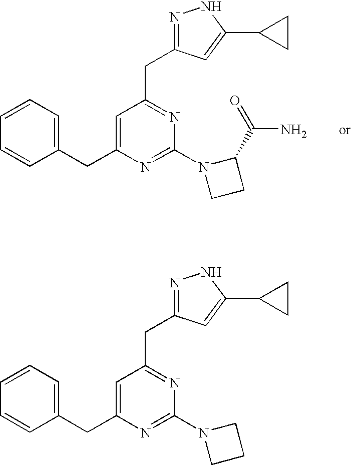

In some embodiments, a PAK inhibitor is a compound of Formula II:

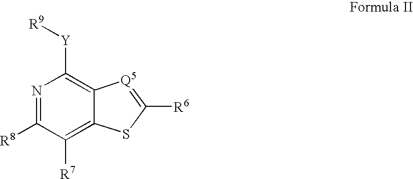

wherein:

R⁶, R⁹ are independently substituted or unsubstituted aryl, substituted or unsubstituted heteroaryl, substituted or unsubstituted cycloalkyl, or substituted or unsubstituted heterocycloalkyl;

R⁷ is H, halo, hydroxy, cyano, substituted or unsubstituted alkyl, substituted or unsubstituted alkoxy, substituted or unsubstituted alkylamino, C(═O)—N(R¹⁰)₂, S(O)ₙ—N(R¹⁰)₂, wherein each R¹⁰ is independently H, substituted or unsubstituted alkyl; substituted or unsubstituted cycloalkyl, or substituted or unsubstituted alkylcycloalkyl; and n is 1-2;
R⁸ is substituted or unsubstituted alkyl, substituted or unsubstituted alkoxy, substituted or unsubstituted alkylamino, substituted or unsubstituted heteroalkyl, substituted or unsubstituted aryl, substituted or unsubstituted heteroaryl, substituted or unsubstituted cycloalkyl, or substituted or unsubstituted heterocycloalkyl;
   Q⁵ is N or C—R¹¹; wherein R¹¹ is H, halo, hydroxy, cyano, substituted or unsubstituted alkyl; or substituted or unsubstituted alkoxy; and
Y is O, N—R¹² or C(R¹²)₂, wherein each R¹² is independently H, hydroxy, substituted or unsubstituted alkyl; or two R¹² taken together are (═O) or (═NR¹³); wherein R¹³ is H, hydroxy, substituted or unsubstituted alkyl, or substituted or unsubstituted alkoxy;
   or a pharmaceutically acceptable salt thereof.

In some embodiments, Y is N—R¹². In some embodiments, Y is NH. In some embodiments, Q⁵ is N, or CH. In some embodiments, Q⁵ is CH.

In some embodiments, R⁶ is substituted or unsubstituted aryl, substituted or unsubstituted heteroaryl, substituted or unsubstituted cycloalkyl, or substituted or unsubstituted heterocycloalkyl. In some embodiments, R⁶ is substituted or unsubstituted aryl, substituted or unsubstituted heteroaryl. In some embodiments, R⁶ is substituted or unsubstituted phenyl, pyridinyl, pyrimidinyl, pyrazinyl, thienyl, pyrazolyl, imidazolyl, triazolyl, oxazolyl, isoxazolyl, or thiazolyl.

In some embodiments, R⁷ is H, halo, hydroxy, cyano, C(═O)—N(R¹⁰)₂, or S(O)ₙ—N(R¹⁰)₂, wherein each R¹⁰ is independently H, substituted or unsubstituted alkyl; substituted or unsubstituted cycloalkyl, or substituted or unsubstituted alkylcycloalkyl; In some embodiments, R⁷ is C(═O)—N(R¹⁰)₂, or S(O)ₙ—N(R¹⁰)₂, wherein each R¹⁰ is independently H. In some embodiments, R⁸ is H, halo, hydroxy, cyano, substituted or unsubstituted alkoxy, or substituted or unsubstituted alkylamine. In some embodiments, R⁸ is H, halo, hydroxy or cyano. In some embodiments, R⁹ is substituted or unsubstituted aryl, substituted or unsubstituted heteroaryl, substituted or unsubstituted cycloalkyl, or substituted or unsubstituted heterocycloalkyl.

In some embodiments, R⁹ is substituted or unsubstituted aryl, substituted or unsubstituted heteroaryl. In some embodiments, R⁹ is substituted or unsubstituted cycloalkyl, or substituted or unsubstituted heterocycloalkyl. In some embodiments, R⁹ is substituted or unsubstituted cyclopropane, substituted or unsubstituted cyclobutane, substituted or unsubstituted cyclopentane, substituted or unsubstituted cyclohexane, substituted or unsubstituted pyran, substituted or unsubstituted tetrahydrofuran, substituted or unsubstituted azetidine, substituted or unsubstituted azetidine, substituted or unsubstituted oxetane, substituted or unsubstituted azetidinone, substituted or unsubstituted oxetone, substituted or unsubstituted pyrrolidine, substituted or unsubstituted piperidine, substituted or unsubstituted pyrrolidinone or substituted or unsubstituted piperidinone

In some embodiments, a compound of Formula II has the structure:

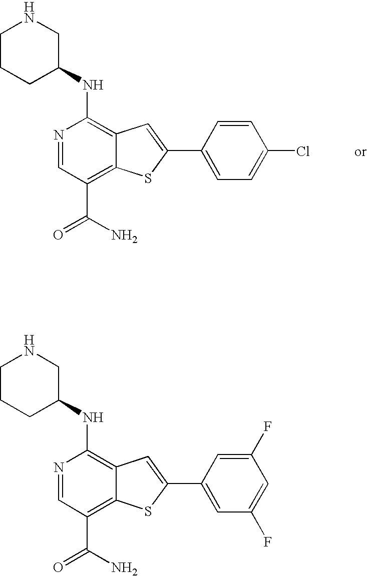

In some embodiments, a PAK inhibitor is a compound of Formula III:

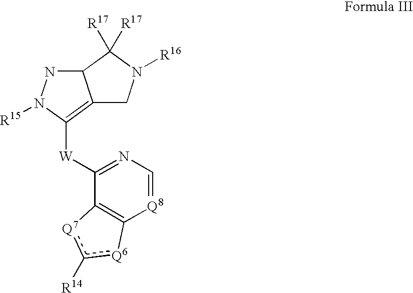

wherein:

R¹⁴ is H, halo, hydroxy, cyano, substituted or unsubstituted alkyl, substituted or unsubstituted alkoxy, substituted or unsubstituted alkylamine, substituted or unsubstituted heteroalkyl, C(═O)—N(R¹⁸)₂, or S(O)ₙ—N(R¹⁸)₂; wherein R¹⁸ is H, or substituted or unsubstituted alkyl;

R¹⁵, R¹⁶ are independently H, substituted or unsubstituted alkyl, C(═O)—N(R¹⁸)₂, or S(O)ₙ—N(R¹⁸)₂;

each R¹⁷ is independently H, halo, hydroxy, cyano, substituted or unsubstituted alkyl, substituted or unsubstituted alkoxy; or two R¹⁷ together are (═O) or (═NR¹⁹); wherein R¹⁹ is H, hydroxy, or substituted or unsubstituted alkyl;
Q⁶, Q⁷, are independently N, S, or C—R²⁰; wherein R²⁰ is H, halo, hydroxy, cyano, substituted or unsubstituted alkyl; or substituted or unsubstituted alkoxy;
   Q8 is N, or C—R²⁰;

W is O, N—R²¹ or C(R²¹)₂, wherein each R²¹ is independently H, hydroxy, substituted or unsubstituted alkyl; or two R²¹ taken together are (═O) or (═NR²²); wherein R²² is H, hydroxy, substituted or unsubstituted alkyl, or substituted or unsubstituted alkoxy;
or a pharmaceutically acceptable salt thereof.

In some embodiments, W is N—R²¹. In some embodiments W is NH. In some embodiments W is O. In some embodiments W is C═NH.

In some embodiments one of Q⁶ and Q⁷ is S and the other is CH. In some embodiments, Q⁶ is S. In some embodiments, Q⁷ is S. In some embodiments Q⁸ is N or CH. In some embodiments Q⁸ is N. In some embodiments R¹⁴ is H, halo, or substituted or unsubstituted alkyl. In some embodiments, R¹⁴ is C(═O)—N(R¹⁸)₂, or S(O)ₙ—N(R¹⁸)₂ and R¹⁸ is H. In some embodiments R¹⁵ is H, or substituted or unsubstituted alkyl. In some embodiments R¹⁶ is H, or substituted or unsubstituted alkyl. In some embodiments R¹⁶ is, C(═O)—N(R¹⁸)₂, or S(O)ₙ—N(R¹⁸)₂ and R¹⁸ is substituted alkyl. In some embodiments R¹⁷ is H, substituted or unsubstituted alkyl; or two R¹⁷ together are (═O).

In some embodiments, a PAK inhibitor is a compound of structure:

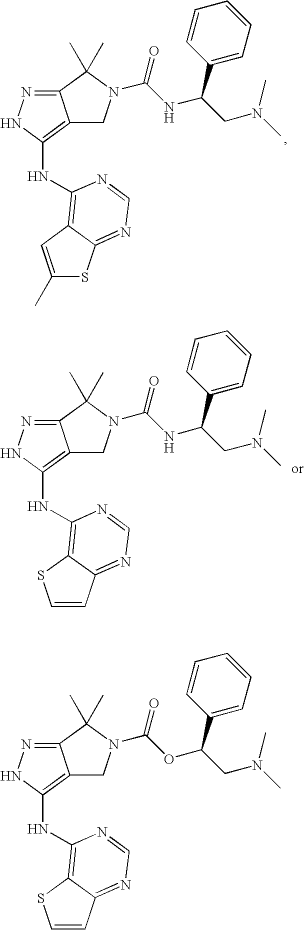

In certain instances, a PAK inhibitor is BMS-387032; SNS-032; CHI4-258; TKI-258; EKB-569; JNJ-7706621; PKC-412; staurosporine; N-[2-(diethylamino)ethyl]-5-[(Z)-(5-fluoro-1,2-dihydro-2-oxo-3H-indol-3-ylidine)methyl]-2,4-dimethyl-1H-pyrrole-3-carboxamide (SU-14813; sunitimb); N-(3-chloro-4-fluoro-phenyl)-7-methoxy-6-(3-morpholin-4-ylpropoxy)quinazolin-4-amine (ZD1839, gefitinib), VX-680; MK-0457; (S)-1-(4-benzyl-6-((5-cyclopropyl-1H-pyrazol-3-yl)methyl)pyrimidin-2-yl)azetidine-2-carboxamide (Compound 1), (S)-2-(3,5-difluorophenyl)-4-(piperidin-3-ylamino)thieno[3,2-c]pyridine-7-carboxamide (Compound 2), combinations thereof; and/or derivatives or analogs or thereof.

PAK inhibitors include, e.g., those disclosed in U.S. Pat. Nos. 5,863,532, 6,191,169, and 6,248,549; U.S. Patent Applications 200200045564, 20020086390, 20020106690, 20020142325, 20030124107, 20030166623, 20040091992, 20040102623, 20040208880, 200500203114, 20050037965, 20050080002, and 20050233965, 20060088897; EP Patent Publication 1492871; PCT patent publication WO 9902701; Kumar et al., (2006), Nat. Rev. Cancer, 6:459; and Eswaran et al., (2007), Structure, 15:201-213.

In some embodiments, a PAK inhibitor is a small molecule. As referred to herein, a “small molecule” is an organic molecule that is less than about 5 kilodaltons (Kd) in size. In some embodiments, the small molecule is less than about 4 Kd, 3 Kd, about 2 Kd, or about 1 Kd. In some embodiments, the small molecule is less than about 800 daltons (D), about 600 D, about 500 D, about 400 D, about 300 D, about 200 D, or about 100 D. In some embodiments, a small molecule is less than about 4000 g/mol, less than about 3000 g/mol, 2000 g/mol, less than about 1500 g/mol, less than about 1000 g/mol, less than about 800 g/mol, or less than about 500 g/mol. In some embodiments, small molecules are non-polymeric. Typically, small molecules are not proteins, polypeptides, polynucleotides, oligonucleotides, polysaccharides, glycoproteins, or proteoglycans, but includes peptides of up to about 40 amino acids. A derivative of a small molecule refers to a molecule that shares the same structural core as the original small molecule, but which is prepared by a series of chemical reactioFns from the original small molecule. As one example, a pro-drug of a small molecule is a derivative of that small molecule. An analog of a small molecule refers to a molecule that shares the same or similar structural core as the original small molecule, and which is synthesized by a similar or related route, or art-recognized variation, as the original small molecule.

Small molecule PAK inhibitors are optionally identified in high-throughput in vitro or cellular assays as described in, e.g., Yu et al (2001), J Biochem (Tokyo); 129(2):243-251; Rininsland et al (2005), BMC Biotechnol, 5:16; and Allen et al (2006), ACS Chem Biol; 1(6):371-376. Direct PAK inhibitors suitable for the methods described herein are available from a variety of sources including both natural (e.g., plant extracts) and synthetic. For example, candidate PAK inhibitors are isolated from a combinatorial library, i.e., a collection of diverse chemical compounds generated by either chemical synthesis or biological synthesis by combining a number of chemical “building blocks.” For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks, as desired. Indeed, theoretically, the systematic, combinatorial mixing of 100 interchangeable chemical building blocks results in the synthesis of 100 million tetrameric compounds or 10 billion pentameric compounds. See Gallop et al., (1994), J. Med. Chem. 37(9), 1233. Preparation and screening of combinatorial chemical libraries are documented methodologies. Combinatorial chemical libraries include, but are not limited to: diversomers such as hydantoins, benzodiazepines, and dipeptides, as described in, e.g., Hobbs et al. (1993), Proc. Natl. Acad. Sci. U.S.A. 90, 6909; analogous organic syntheses of small compound libraries, as described in Chen et al. (1994), J. Amer. Chem. Soc., 116: 2661; Oligocarbamates, as described in Cho, et al. (1993), Science 261, 1303; peptidyl phosphonates, as described in Campbell at al. (1994), J. Org. Chem., 59: 658; and small organic molecule libraries containing, e.g., thiazolidinones and metathiazanones (U.S. Pat. No. 5,549,974), pyrrolidines (U.S. Pat. Nos. 5,525,735 and 5,519,134), benzodiazepines (U.S. Pat. No. 5,288,514). In addition, numerous combinatorial libraries are commercially available from, e.g., ComGenex (Princeton, N.J.); Asinex (Moscow, Russia); Tripos, Inc. (St. Louis, Mo.); ChemStar, Ltd. (Moscow, Russia); 3D Pharmaceuticals (Exton, Pa.); and Martek Biosciences (Columbia, Md.).

Devices for the preparation of combinatorial libraries are commercially available (see, e.g., 357 MPS, 390 MPS from Advanced Chem Tech, Louisville, Ky.; Symphony from Rainin, Woburn, Mass.; 433A from Applied Biosystems, Foster City, Calif.; and 9050 Plus from Millipore, Bedford, Mass.). A number of robotic systems have also been developed for solution phase chemistries. These systems include automated workstations like the automated synthesis apparatus developed by Takeda Chemical Industries, LTD (Osaka, Japan), and many robotic systems utilizing robotic arms (Zymate II, Zymark Corporation, Hopkinton, Mass.; Orca, Hewlett Packard, Palo Alto, Calif.) which mimic the manual synthetic operations performed by a chemist. Any of the above devices are optionally used to identify and characterize small molecule PAK inhibitors suitable for the methods disclosed herein. In many of the embodiments disclosed herein, PAK inhibitors, PAK binding molecules, and PAK clearance agents are disclosed as polypeptides or proteins (where polypeptides comprise two or more amino acids). In these embodiments, the inventors also contemplate that PAK inhibitors, binding molecules, and clearance agents also include peptide mimetics based on the polypeptides, in which the peptide mimetics interact with PAK or its upstream or downstream regulators by replicating the binding or substrate interaction properties of PAK or its regulators. Nucleic acid aptmers are also contemplated as PAK inhibitors, binding molecules, and clearance agents, as are small molecules other than peptides or nucleic acids. For example, in some embodiments small molecule PAK binding partners, inhibitors, or clearance agents, or small molecule agonists or antagonists of PAK modulators or targets, are designed or selected based on analysis of the structure of PAK or its modulators or targets and binding interactions with interacting molecules, using “rational drug design” (see, for example Jacobsen et al. (2004) Molecular Interventions 4:337-347; Shi et al. (2007) Bioorg. Med. Chem. Lett. 17:6744-6749).

Detection of PAK dependent phosphorylation of a substrate can be quantified by a number of means other than measurement of radiolabeled phosphate incorporation. For example, incorporation of phosphate groups may affect physiochemical properties of the substrate such as electrophoretic mobility, chromatographic properties, light absorbance, fluorescence, and phosphorescence. Alternatively, monoclonal or polyclonal antibodies can be generated which selectively recognize phosphorylated forms of the substrate from non-phosphorylated forms whereby allowing antibodies to function as an indicator of PAK kinase activity.

High-throughput PAK kinase assays can be perfumed in, for example, microliter plates with each well containing PAK kinase or an active fragment thereof, substrate covalently linked to each well, P³² radiolabled ATP and a potential PAK inhibitor candidate. Microtiter plates can contain 96 wells or 1536 wells for large scale screening of combinatorial library compounds. After the phosphorylation reaction has completed, the plates are washed leaving the bound substrate. The plates are then detected for phosphate group incorporation via autoradiography or antibody detection. Candidate PAK inhibitors are identified by their ability to decease the amount of PAK phosphotransferase ability upon a substrate in comparison with PAK phosphotransferase ability alone.

The identification of potential PAK inhibitors may also be determined, for example, via in vitro competitive binding assays on the catalytic sites of PAK such as the ATP binding site and/or the substrate binding site. For binding assays on the ATP binding site, a known protein kinase inhibitor with high affinity to the ATP binding site is used such as staurosporine. Staurosporine is immobolized and may be fluorescently labeled, radiolabeled or in any manner that allows detection. The labeled staurosporine is introduced to recombinantly expressed PAK protein or a fragment thereof along with potential PAK inhibitor candidates. The candidate is tested for its ability to compete, in a concentration-dependant manner, with the immobolized staurosporine for binding to the PAK protein. The amount of staurosporine bound PAK is inversely proportional to the affinity of the candidate inhibitor for PAK. Potential inhibitors would decrease the quantifiable binding of staurosporine to PAK. See e.g., Fabian et al (2005) Nat. Biotech., 23:329. Candidates identified from this competitive binding assay for the ATP binding site for PAK would then be further screened for selectivity against other kinases for PAK specificity.

The identification of potential PAK inhibitors may also be determined, for example, by in cyto assays of PAK activity in the presence of the inhibitor candidate. Various cell lines and tissues may be used, including cells specifically engineered for this purpose. In cyto screening of inhibitor candidates may assay PAK activity by monitoring the downstream effects of PAK activity. Such effects include, but are not limited to, the formation of peripheral actin microspikes and or associated loss of stress fibers as well as other cellular responses such as growth, growth arrest, differentiation, or apoptosis. See e.g., Zhao et al., (1998) Mol. Cell. Biol. 18:2153. For example in a PAK yeast assay, yeast cells grow normally in glucose medium. Upon exposure to galactose however, intracellular PAK expression is induced, and in turn, the yeast cells die. Candidate compounds that inhibit PAK activity are identified by their ability to prevent the yeast cells from dying from PAK activation.

Alternatively, PAK-mediated phosphorylation of a downstream target of PAK can be observed in cell based assays by first treating various cell lines or tissues with PAK inhibitor candidates followed by lysis of the cells and detection of PAK mediated events. Cell lines used in this experiment may include cells specifically engineered for this purpose. PAK mediated events include, but are not limited to, PAK mediated phosphorylation of downstream PAK mediators. For example, phosphorylation of downstream PAK mediators can be detected using antibodies that specifically recognize the phosphorylated PAK mediator but not the unphosphorylated form. These antibodies have been described in the literature [insert reference] and have been extensively used in kinase screening campaigns.

The identification of potential PAK inhibitors may also be determined, for example, by in vivo assays involving the use of animal models, including transgenic animals that have been engineered to have specific defects or carry markers that can be used to measure the ability of a candidate substance to reach and/or affect different cells within the organism. For example, fragile X mental retardation 1 (FMR1) knockout mice reportedly have defects in synaptic plasticity and behavior from increased numbers of dendritic spines and an abundance of long and immature spines. See e.g., Comery et al., (1997) Proc. Natl. Acad. Sci. USA, 94:5401-04. As PAK is a downstream effector of the FMR1 gene, the defects are reversed upon the use of dominant negative transgenes of PAK that inhibit endogenous PAK activity. See Hayashi et al. (2007) Proc. Natl. Acad. Sci. USA, 104:11489-94. Thus, identification of PAK inhibitors can comprise administering a candidate to FMR1 knockout mice and observing for reversals in synaptic plasticity and behavior defects as a readout for PAK inhibition. Administration of the candidate to the animal is via any clinical or non-clinical route, including but not limited to oral, nasal, buccal and/or topical adiminstrations. Additionally or alternatively, administration may be intratracheal instillation, bronchial instillation, intradermal, subcutaneous, intramuscular, intraperitoneal, inhalation, and/or intravenous injection.

Changes in spine morphology are detected using any suitable method, e.g., by use of 3D and/or 4D real time interactive imaging and visualization. In some instances, the Imaris suite of products (available from Bitplane Scientific Solutions) provides functionality for visualization, segmentation and interpretation of 3D and 4D microscopy datasets obtained from confocal and wide field microscopy data.

In some embodiments, a PAK inhibitor suitable for the methods described herein decreases PAK activity relative to a basal level of PAK activity by about 1.1 fold to about 100 fold, e.g., to about 1.2 fold, 1.5 fold, 1.6 fold, 1.7 fold, 2.0 fold, 3.0 fold, 5.0 fold, 6.0 fold, 7.0 fold, 8.5 fold, 9.7 fold, 10 fold, 12 fold, 14 fold, 15 fold, 20 fold, 30 fold, 40 fold, 50 fold, 60 fold, 70 fold, 90 fold, 95 fold, or by any other amount from about 1.1 fold to about 100 fold relative to basal PAK activity. In some embodiments, a PAK inhibitor is a reversible PAK inhibitor. In other embodiments, a PAK inhibitor is an irreversible PAK inhibitor. Direct PAK inhibitors are optionally used for the manufacture of a medicament for treating any of the NCs described herein (e.g., psychotic disorders, mood disorders, age-related cognitive decline, epilepsy, Huntington's Disease, or Parkinson's Disease).

In some embodiments, a PAK inhibitor suitable for the methods described herein modulates a spine:head ratio, e.g., ratio of the volume of the spine to the volume of the head, ratio of the length of a spine to the length of a head of the spine, ratio of the surface area of a spine to the surface area of the head of a spine, or the like, compared to a spine:head ratio in the absence of a PAK inhibitor. In certain embodiments, a PAK inhibitor suitable for the methods described herein modulates the volume of the spine head, the width of the spine bead, the surface area of the spine head, the length of the spine shaft, the diameter of the spine shaft, or a combination thereof. In some embodiments, provided herein is a method of modulating the volume of a spine head, the width of a spine head, the surface area of a spine head, the length of a spine shaft, the diameter of a spine shaft, or a combination thereof, by contacting a neuron comprising the dendritic spine with an effective amount of a PAK inhibitor described herein. In specific embodiments, the neuron is contacted with the PAK inhibitor in vivo. FIG. 4 illustrates various morphologies of the dendritic spines.

In some embodiments, a PAK inhibitor used for the methods described herein has in vitro ED₅₀ for PAK activation of less than 100 μM (e.g., less than 10 μM, less than 5 μM, less than 4 μM, less than 3 μM, less than 1 μM, less than 0.8 μM, less than 0.6 μM, less than 0.5 μM, less than 0.4 μM, less than 0.3 μM, less than less than 0.2 μM, less than 0.1 μM, less than 0.08 μM, less than 0.06 μM, less than 0.05 μM, less than 0.04 μM, less than 0.03 μM, less than less than 0.02 μM, less than 0.01 μM, less than 0.0099 μM, less than 0.0098 μM, less than 0.0097 μM, less than 0.0096 μM, less than 0.0095 μM, less than 0.0094 μM, less than 0.0093 μM, less than 0.00092, or less than 0.0090 μM).

In some embodiments, exemplary small molecule PAK inhibitors that are used in accordance with methods described herein include BMS-387032; SNS-032; CHI4-258; TKI-258; EKB-569; NJ-7706621; PKC-412; staurosporine; SU-14813; sunitinib; VX-680; MK-0457; combinations thereof; and/or derivatives analogs or thereof.

In some embodiments, a PAK inhibitor is a polypeptide comprising an amino acid sequence about 80% to about 100% identical, e.g., 85%, 90%, 92%, 93%, 95%, 96%, 97%, 98%, 99%, or any other percent from about 80% to about 100% identical the following amino acid sequence: HTIHVGFDAVTGEFTGMPEQWARLLQTSNITKSEQKKNPQAVLDVLEFYNSKKTSNSQ KYMSFTDKS

The above sequence corresponds to the PAK autoinhibitory domain (PAD) polypeptide amino acids 83-149 of PAK1 polypeptide as described in, e.g., Zhao et al (1998). In some embodiments, a PAK inhibitor is a fusion protein comprising the above-described PAD amino acid sequence. In some embodiments, in order to facilitate cell penetration the fusion polypeptide (e.g., N-terminal or C-terminal) further comprises a polybasic protein transduction domain (PTD) amino acid sequence, e.g.: RKKRRQRR; YARAAARQARA; THRLPRRRRRR; or GGRRARRRRRR.

In some embodiments, in order to enhance uptake into the brain, the fusion polypeptide further comprises a human insulin receptor antibody as described in U.S. patent application Ser. No. 11/245,546.

In some embodiments, a PAK inhibitor is peptide inhibitor comprising a sequence at least 60% to 100%, e.g., 65%, 70%, 75%, 80%, 85%, 90%, 92%, 93%, 95%, 96%, 97%, 98%, 99%, or any other percent from about 60% to about 100% identical the following amino acid sequence: PPVIAPREHTKSVYTRS as described in, e.g., Zhao et al (2006), Nat Neurosci, 9(2):234-242. In some embodiments, the peptide sequence further comprises a PTD amino acid sequence as described above.

In some embodiments, a PAK inhibitor is a polypeptide comprising an amino acid sequence at least 80% to 100%, e.g., 85%, 90%, 92%, 93%, 95%, 96%, 97%, 98%, 99%, or any other percent from about 80% to about 100% identical to the FMRP1 protein (GenBank Accession No. Q06787), where the polypeptide is able to bind with a Group I PAK (for example, PAK1, PAK2, and/or PAK3). In some embodiments, a PAK inhibitor is a polypeptide comprising an amino acid sequence at least 80% to 100%, e.g., 85%, 90%, 92%, 93%, 95%, 96%, 97%, 98%, 99%, or any other percent from about 80% to about 100% identical to the FMRP1 protein (GenBank Accession No. Q06787), where the polypeptide is able to bind with a Group I PAK, such as, for example PAK1 (see, e.g., Hayashi et al (2007), Proc Natl Acad Sci USA, 104(27):11489-11494. In some embodiments, a PAK inhibitor is a polypeptide comprising a fragment of human FMRP1 protein with an amino acid sequence at least 80% to 100%, e.g., 85%, 90%, 92%, 93%, 95%, 96%, 97%, 98%, 99%, or any other percent from about 80% to about 100% identical to the sequence of amino acids 207-425 of the human FMRP1 protein (i.e., comprising the KH1 and KH2 domains), where the polypeptide is able to bind to PAK1.

In some embodiments, a PAK inhibitor comprises a polypeptide comprising an amino acid sequence at least 80% to 100%, e.g., 85%, 90%, 92%, 93%, 95%, 96%, 97%, 98%, 99%, or any other percent from about 80% to about 100% identical to at least five, at least ten at least twenty, at least thirty, at least forty, at least fifty, at least sixty, at least seventy, at least eighty, at least ninety contiguous amino acids of the huntingtin (htt) protein (GenBank Accession No. NP 002102, gi 90903231), where the polypeptide is able to bind to a Group 1 PAK (for example, PAK1, PAK2, and/or PAK3). In some embodiments, a PAK inhibitor comprises a polypeptide comprising an amino acid sequence at least 80% to 100%, e.g., 85%, 90%, 92%, 93%, 95%, 96%, 97%, 98%, 99%, or any other percent from about 80% to about 100% identical to at least a portion of the huntingtin (hit) protein (GenBank Accession No. NP 002102, gi 90903231), where the polypeptide is able to bind to PAK1. In some embodiments, a PAK inhibitor is a polypeptide comprising a fragment of human huntingtin protein with an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the human huntingtin protein that is outside of the sequence encoded by exon 1 of the htt gene (i.e., a fragment that does not contain poly glutamate domains), where the polypeptide binds a Group I PAK. In some embodiments, a PAK inhibitor is a polypeptide comprising a fragment of human huntingtin protein with an amino acid sequence at least 80% identical to a sequence of the human huntingtin protein that is outside of the sequence encoded by exon 1 of the htt gene (i.e., a fragment that does not contain poly glutamate domains), where the polypeptide binds PAK1.

In some embodiments, a PAK inhibitor polypeptide is delivered to one or more brain regions of a subject by administration of a viral expression vector, e.g., an AAV vector, a lentiviral vector, an adenoviral vector, or a HSV vector. A number of viral vectors for delivery of therapeutic proteins are described in, e.g., U.S. Pat. Nos., 7,244,423, 6,780,409, 5,661,033. In some embodiments, a PAK inhibitor polypeptide to be expressed is under the control of an inducible promoter (e.g., a promoter containing a tet-operator). Inducible viral expression vectors include, for example, those disclosed in U.S. Pat. No. 6,953,575. Inducible expression of a PAK inhibitor polypeptide allows for tightly controlled and reversible increases of PAK inhibitor polypeptide expression by varying the dose of an inducing agent (e.g., tetracycline) administered to the subject.

In some embodiments, PAK inhibitors act by decreasing transcription and/or translation of PAK. A PAK inhibitor in some embodiments decreases transcription and/or translation of a Group I PAK. For example, in some embodiments, modulation of PAK transcription or translation occurs through the administration of specific or non-specific inhibitors of PAK transcription or translation. In some embodiments, proteins or non-protein factors that bind the upstream region of the PAK gene or the 5′ UTR of a PAK mRNA are assayed for their affect on transcription or translation using transcription and translation assays (see, for example, Baker, et al. (2003) J. Biol. Chem. 278: 17876-17884; Jiang et al. (2006) J Chromatography A 1133: 83-94; Novoa at al. (1997) Biochemistry 36: 7802-7809; Brandi at al. (2007) Methods Enzymol. 431: 229-267). PAK inhibitors include DNA or RNA binding proteins or factors that reduce the level of transcription or translation or modified versions thereof. In other embodiments, a PAK inhibitor is a modified form (e.g., mutant form or chemically modified form) of a protein or other compound that positively regulates transcription or translation of PAK, in which the modified form reduces transcription or translation of PAK. In yet other embodiments, a transcription or translation inhibitor is an antagonist of a protein or compound that positively regulates transcription or translation of PAK, or is an agonist of a protein that represses transcription or translation.

Regions of a gene other than those upstream of the transcriptional start site and regions of an mRNA other than the 5′ UTR (such as but not limited to regions 3′ of the gene or in the 3′ UTR of an mRNA, or regions within intron sequences of either a gene or mRNA) also include sequences to which effectors of transcription, translation, mRNA processing, mRNA transport, and mRNA stability bind. In some embodiments, a PAK inhibitor is a clearance agent comprising a polypeptide having homology to an endogenous protein that affects mRNA processing, transport, or stability, or is an antagonist or agonist of one or more proteins that affect mRNA processing, transport, or turnover, such that the inhibitor reduces the expression of PAK protein by interfering with PAK mRNA transport or processing, or by reducing the half-life of PAK mRNA. A PAK clearance agents in some embodiments interferes with transport or processing of a Group I PAK mRNA, or by reducing the half-life of a Group I PAK mRNA.

For example, PAK clearance agents decrease RNA and/or protein half-life of a PAK isoform, for example, by directly affecting mRNA and/or protein stability. In certain embodiments, PAK clearance agents cause PAK mRNA and/or protein to be more accessible and/or susceptible to nucleases, proteases, and/or the proteasome. In some embodiments, PAK inhibitors decrease the processing of PAK mRNA thereby reducing PAK activity. For example, PAK inhibitors function at the level of pre-mRNA splicing, 5′ end formation (e.g. capping), 3′ end processing (e.g. cleavage and/or polyadenylation), nuclear export, and/or association with the translational machinery and/or ribosomes in the cytoplasm. In some embodiments, PAK inhibitors cause a decrease in the level of PAK mRNA and/or protein, the half-life of PAK mRNA and/or protein by at least about 5%, at least about 10%, at least about 20%, at least about 30%, at least about 40%, at least about 50%, at least about 60%, at least about 80%, at least about 90%, at least about 95%, or substantially 100%.

In some embodiments, a PAK inhibitor is a clearance agent that comprises one or more RNAi or antisense oligonucleotides directed against one or more PAK isoform RNAs. In some embodiments, a PAK inhibitor comprises one or more ribozymes directed against one or more PAK isoform RNAs. The design, synthesis, and use of RNAi constructs, antisense oligonucleotides, and ribozymes are found, for example, in Dykxboorn et al. (2003) Nat. Rev. Mol. Cell. Biol. 4: 457-467; Hannon et al. (2004) Nature 431: 371-378; Sarver et al. (1990) Science 247:1222-1225; Been et al. (1986) Cell 47:207-216). In some embodiments, nucleic acid constructs that induce triple helical structures are also introduced into cells to inhibit transcription of the PAK gene (Helene (1991) Anticancer Drug Des. 6:569-584).

For example, a PAK inhibitor that is a clearance agent is in some embodiments an RNAi molecule or a nucleic acid construct that produces an RNAi molecule. An RNAi molecule comprises a double-stranded RNA of at least about seventeen bases having a 2-3 nucleotide single-stranded overhangs on each end of the double-stranded structure, in which one strand of the double-stranded RNA is substantially complementary to the target PAK RNA molecule whose downregulation is desired. “Substantially complementary” means that one or more nucleotides within the double-stranded region are not complementary to the opposite strand nucleotide(s). Tolerance of mismatches is optionally assessed for individual RNAi structures based on their ability to downregulate the target RNA or protein. In some embodiments, RNAi is introduced into the cells as one or more short hairpin RNAs (“shRNAs”) or as one or more DNA constructs that are transcribed to produce one or more shRNAs, in which the shRNAs are processed within the cell to produce one or more RNAi molecules.

Nucleic acid constructs for the expression of siRNA, shRNA, antisense RNA, ribozymes, or nucleic acids for generating triple helical structures are optionally introduced as RNA molecules or as recombinant DNA constructs. DNA constructs for reducing gene expression are optionally designed so that the desired RNA molecules are expressed in the cell from a promoter that is transcriptionally active in mammalian cells, such as, for example, the SV40 promoter, the human cytomegalovirus immediate-early promoter (CMV promoter), or the pol III and/or pol II promoter using known methods. For some purposes, it is desirable to use viral or plasmid-based nucleic acid constructs. Viral constructs include but are not limited to retroviral constructs, lentiviral constructs, or based on a pox virus, a herpes simplex virus, an adenovirus, or an adeno-associated virus (AAV).

In other embodiments, a PAK inhibitor is a polypeptide that decreases the activity of PAK. In some embodiments, a PAK inhibitor is a polypeptide that decreases the activity of a Group I PAK. Protein and peptide inhibitors of PAK are optionally based on natural substrates of PAK, e.g., Myosin light chain kinase (MLCK), regulatory Myosin light chain (R-MLC), Myosins I heavy chain, myosin II heavy chain, Myosin VI, Caldesmon, Desmin, Op18/stathmin, Merlin, Filamin A, LIM kinase (LIMK), Ras, Raf, Mek, p47(phox), BAD, caspase 3, estrogen and/or progesterone receptors, NET1, Gαz, phosphoglycerate mutase-B, RhoGDI, prolactin, p41Arc, and/or Aurora-A. In some embodiments, a PAK inhibitor is based on a sequence of PAK itself, for example, the autoinhibitory domain in the N-terminal portion of the PAK protein that binds the catalytic domain of a partner PAK molecule when the PAK molecule is in its homodimeric state (Zhao et al. (1998) Mol. Cell Biol. 18:2153-2163; Knaus et al. (1998) J. Biol. Chem. 273: 21512-21518; Hofman et al. (2004) J. Cell Sci. 117: 4343-4354). In some embodiments, polypeptide inhibitors of PAK comprise peptide mimetics, in which the peptide has binding characteristics similar to a natural binding partner or substrate of PAK.

In some embodiments, compounds that downregulate PAK protein level activate or increase the activity of an upstream regulator or downstream target of PAK. In some embodiments, compounds that downregulate protein level of a Group I PA reduce at least one of the symptoms related to a pathology, such as a neuropsychiatric disorder or a cognitive disorder, by reducing the amount of PAK in a cell. In some embodiments a compound that decreases PAK protein levels in cells also decreases the activity of PAK in the cells. In some embodiments a compound that decreases PAK protein levels does not have a substantial impact on PAK activity in cells. In some embodiments a compound that increases PAK activity in cells decreases PAK protein levels in the cells.

In some embodiments, a compound that decreases the amount of PAK protein in cells decreases transcription and/or translation of PAK or increases the turnover rate of PAK mRNA or protein by modulating the activity of an upstream effector or downstream regulator of PAK. In some embodiments, PAK expression or PAK levels are influenced by feedback regulation based on the conformation, chemical modification, binding status, or activity of PAK itself. In some embodiments, PAK expression or PAK levels are influenced by feedback regulation based on the conformation, chemical modification, binding status, or activity of molecules directly or indirectly acted on by PAK signaling pathways. As used herein “binding status” refers to any or a combination of whether PAK, an upstream regulator of PAK, or a downstream effector of PAK is in a monomeric state or in an oligomeric complex with itself, or whether it is bound to other polypeptides or molecules. For example, a downstream target of PAK, when phosphorylated by PAK, in some embodiments directly or indirectly downregulates PAK expression or decrease the half-life of PAK mRNA or protein. Downstream targets of PAK include but are not limited to: Myosin light chain kinase (MLCK), regulatory Myosin light chain (R-MLC), Myosins I heavy chain, myosin H heavy chain, Myosin VI, Caldesmon, Desmin, Op18/stathmin, Merlin, Filamin A, LIM kinase (LIMK), Ras, Rat Mek, p47phox, BAD, caspase 3, estrogen and/or progesterone receptors, NET1, Gαz, phosphoglycerate mutase-B, RhoGDI, prolactin, p41Arc, and/or Aurora-A. Downregulators of PAK levels include downstream targets of PAK or fragments thereof in a phosphorylated state and downstream targets of PAK or fragments thereof in a hyperphosphorylated state.

A fragment of a downstream target of PAK includes any fragment with an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the downstream regulator, in which the fragment of the downstream target of PAK is able to downregulate PAK mRNA or protein expression or increase turnover of PAK mRNA or protein. In some embodiments, the fragment of a downstream regulator of PAK comprises a sequence that includes a phosphorylation site recognized by PAK, in which the site is phosphorylated.

A compound that decreases the level of PAK includes a peptide, polypeptide, or small molecule that inhibits dephosphorylation of a downstream target of PAK, such that phosphorylation of the downstream target remains at a level that leads to downregulation of PAK levels.

In other embodiments, a PAK inhibitor affects that activity of a molecule that acts in a signaling pathway upstream of PAK (upstream effectors of PAK). In some embodiments, compounds reducing PAK levels decrease PAK transcription or translation or reduce RNA or protein levels by increasing the activity of an upstream effector of PAK. Upstream effectors of PAK include, but are not limited to: TrkB receptors; NMDA receptors; EphB receptors; FMRP; Rho-family GTPases, including Cdc42, Rae (including but not limited to Rac1 and Rac2), Chp, TC10, Tc1, and Wrnch-1; guanine nucleotide exchange factors (“GEFs”), such as but not limited to GEFT, members of the Dbl family of GEFs, p21-activated kinase interacting exchange factor (PDC), DEF6, Zizimin 1, Vav1, Vav2, Dbs, members of the DOCK180 family, Kalirin-7, and Tiam1; G protein-coupled receptor kinase-interacting protein 1 (GIT1), CIB1, filamin A, PI3 kinase, NCK, GRB2, PDK1, CDK5, Cdc42, Etk/Bmx, p35/Cdk5 kinase, and sphingosine.

In some embodiments, a modulator of an upstream regulator of PAKs is an indirect inhibitor of PAK. In certain instances, a modulator of an upstream regulator of PAKs is a modulator of PDK1. In some instances, a modulator of PDK1 reduces of inhibits the activity of PDK1. In some instances a PDK1 inhibitor is an antisense compound (e.g., any PDK1 inhibitor described in U.S. Pat. No. 6,124,272, which PDK1 inhibitor is incorporated herein by reference). In some instances, a PDK1 inhibitor is a compound described in e.g., U.S. Pat. Nos. 7,344,870, and 7,041,687, which PDK1 inhibitors are incorporated herein by reference. In some embodiments, an indirect inhibitor of PAK is a modulator of a PI3 kinase. In some instances a modulator of a P13 kinase is a PI3 kinase inhibitor. In some instances, a PI3 kinase inhibitor is an antisense compound (e.g., any PI3 kinase inhibitor described in WO 2001/018023, which PI3 kinase inhibitors are incorporated herein by reference). In some instances, an inhibitor of a PI3 kinase is 3-morpholino-5-phenylnaphthalen-1(4H)-one (LY294002), or a peptide based covalent conjugate of LY294002, (e.g., SF1126, Semaphore pharmaceuticals). In certain embodiments, an indirect inhibitor of PAK is a modulator of Cdc42. In certain embodiments, a modulator of Cdc42 is an inhibitor of Cdc42. In certain embodiments, a Cdc42 inhibitor is an antisense compound (e.g., any Cdc42 inhibitor described in U.S. Pat. No. 6,410,323, which Cdc42 inhibitors are incorporated herein by reference). In some instances, an indirect inhibitor of PAK is a modulator of GRB2. In some instances, a modulator of GRB2 is an inhibitor of GRB2. In some instances a GRB2 inhibitor is a GRb2 inhibitor described in e.g., U.S. Pat. No. 7,229,960, which GRB2 inhibitor is incorporated by reference herein. In certain embodiments, an indirect inhibitor of PAK is a modulator of NCK. In certain embodiments, an indirect inhibitor of PAK is a modulator of ETK. In some instances, a modulator of ETK is an inhibitor of ETK. In some instances an ETK inhibitor is a compound e.g., [symbol] -Cyano-(3,5-di-t-butyl-4-hydroxy)thiocinnamide (AG 879)

In some embodiments, a compound that decreases PAK levels is an upstream effector of PAK, in which the upstream effector is overexpressed in target cells. In some embodiments, a compound that decreases PAK levels is an upstream effector of a Group I PAK that is overexpressed in target cells. Without limiting PAK inhibitors or clearance agents or methods of their use to any particular mechanism, it has been observed that exogenous expression of the activated forms of the Rho family GTPases Chp and cdc42 in cells leads to increased activation of PAK while at the same time increasing turnover of the PAK protein, significantly lowering its level in the cell (Hubsman et al. (2007) Biochem. J. 404: 487-497). PAK clearance agents thus include agents that increase expression of one or more Rho family GTPases and/or one or more guanine nucleotide exchange factors (GEFs) that regulate the activity of Rho family GTPases, in which overexpression of a Rho family GTPase and/or a GEF results in lower levels of PAK protein in cells. PAK clearance agents also include agonists of Rho family GTPases, as well as agonists of GTP exchange factors that activate Rho family GTPases, such as but not limited to agonists of GEFs of the Dbl family that activate Rho family GTPases.

Overexpression of a Rho family GTPase is optionally by means of introducing a nucleic acid expression construct into the cells or by administering a compound that induces transcription of the endogenous gene encoding the GTPase. In some embodiments, the Rho family GTPase is Rae (e.g., Rac1, Rac2, or Rac3), cdc42, Chp, TC10, Tc1, or Wrnch-1. For example, a Rho family GTPase includes Rac1, Rac2, Rac3, or cdc42. A gene introduced into cells that encodes a Rho family GTPase optionally encodes a mutant form of the gene, for example, a more active form (for example, a constitutively active form, Hubsman et al. (2007) Biochem. J. 404: 487-497). In some embodiments, a PAK clearance agent is, for example, a nucleic acid encoding a Rho family GTPase, in which the Rho family GTPase is expressed from a constitutive or inducible promoter. PAK levels in some embodiments are reduced by a compound that directly or indirectly enhances expression of an endogenous gene encoding a Rho family GTPase.

A PAK clearance agent in some embodiments is a Rho family GTPase agonist, or is a compound that directly or indirectly increases the activation level of one or more Rho family GTPases. In some embodiments a PAK clearance agent is a compound that increases the level of an activated Rho family GTPase, such as, but not limited to, Rac or cdc42. The compound is, as nonlimiting examples, a compound that modifies a Rho family GTPase such that it is constitutively activated, or a compound that binds or modifies a Rho family GTPase to increase the longevity or stability of its activated (GTP bound) state. Activating mutations of Rho family GTPases are known (Hubsman et al. (2007) Biochem. J. 404: 487-497), as are bacterial toxins such as E. coli necrotizing factors 1 and 2 (CNF1 and CNF2) and Bordetella bronchiseptica dermonecrotizing toxin (DNT) that modify Rho family GTPases to cause their constitutive activation (Fiorentini et al. (2003) Cell Death and Differentiation 10:147-152). Toxins such as CNF1, CNF2, and DNT, fragments thereof that increase the activity of a Rho family GTAPase, or peptides or polypeptides that increase the activity of a Rho family GTAPase having an amino acid sequence at least 80% to 100%, e.g., 85%, 90%, 92%, 93%, 95%, 96%, 97%, 98%, 99%, or any other percent from about 80% to about 100% identical to a sequence of at least ten, at least twenty, at least thirty, at least forty, at least fifty, at least sixty, at least seventy, at least eighty, at least ninety, or at least 100 contiguous amino acids of the toxin are also used as PAK clearance agents. Small molecule inhibitors designed to mimic the effect of activating mutations of GTPases that are upstream regulators of PAK or designed to mimic the effect of bacterial toxins that activate GTPases that bind and activate PAK are also included as compounds that downregulate PAK levels.

In some embodiments, the inhibitor is a compound that inhibits post-translational modification of a Rho family GTPase. For example, in some embodiments a compound that inhibits prenylation of small Rho-family GTPases such as Rho, Rac, and cdc42 is used to increase GTPase activity and thereby reduce the amount of PAK in the cell. In some embodiments, a compound that decreases PAK levels is a bisphosphonate compound that inhibits prenylation of Rho-family GTPases such as cdc42 and Rac, in which nonprenylated GTPases have higher activity than their prenylated counterparts (Dunford et al. (2006) J. Bone Miner. Res. 21: 684-694; Reszka at al. (2004) Mini Rev. Med. Chem. 4: 711-719).

In some embodiments a compound that decreases PAK levels in the cell is a compound that directly or indirectly increases the activity of a guanine exchange factor (GEF) that promotes the active state of a Rho family GTPase, such as an agonist of a GEF that activates a Rho family GTPase, such as but not limited to, Rac or cdc42. Activation of GEFs is also effected by compounds that activate TrkB, NMDA, or EphB receptors.

In some embodiments, a PAK clearance agent is a nucleic acid encoding a GEF that activates a Rho family GTPase, in which the GEF is expressed from a constitutive or inducible promoter. In some embodiments, a guanine nucleotide exchange factor (GEF), such as but not limited to a GEF that activates a Rho family GTPase is overexpressed in cells to increase the activation level of one or more Rho family GTPases and thereby lower the level of PAK in cells. GEFs include, for example, members of the Dbl family of GTPases, such as but not limited to, GEFT, PIX (e.g., alphaPIX, betaPIX), DEF6, Zizimin 1, Vav1, Vav2, Dbs, members of the DOCK180 family, hPEM-2, FLJ00018, kalirin, Tiam1, STEF, DOCK2, DOCK6, DOCK7, DOCK9, Asf, EhGEF3, or GEF-1. In some embodiments, PAK levels are also reduced by a compound that directly or indirectly enhances expression of an endogenous gene encoding a GEF. A GEF expressed from a nucleic acid construct introduced into cells is in some embodiments a mutant GEF, for example a mutant having enhanced activity with respect to wild type.

The clearance agent is optionally a bacterial toxin such as Salmonella typhinmurium toxin SpoE that acts as a GEF to promote cdc42 nucleotide exchange (Buchwald et al. (2002) EMBO J. 21: 3286-3295; Schlumberger et al. (2003) J. Biological Chem. 278: 27149-27159). Toxins such as SopE, fragments thereof or peptides or polypeptides having an amino acid sequence at least 80% to 100%, e.g., 85%, 90%, 92%, 93%, 95%, 96%, 97%, 98%, 99%, or any other percent from about 80% to about 100% identical to a sequence of at least five, at least ten, at least twenty, at least thirty, at least forty, at least fifty, at least sixty, at least seventy, at least eighty, at least ninety, or at least 100 contiguous amino acids of the toxin are also optionally used as downregulators of PAK activity. The toxin is optionally produced in cells from nucleic acid constructs introduced into cells.

In some embodiments a PAK inhibitors, binding molecules, and clearance agents provided herein are administered to a subject with a neuropsychiatric condition or predicted to develop a neuropsychiatric condition to delay the loss of dendritic spine density in the subject A pharmacological composition comprising a therapeutically effective amount of at least one of the compounds disclosed herein, including: a PAK transcription inhibitor, a PAK clearance agent, an agent that binds PAK to prevent its interaction with one or more cellular or extracellular proteins, and a PAK antagonist. In some preferred embodiments, the pharmacological composition comprises a therapeutically effective amount of at least one of the compounds chosen from the group consisting of: a Group 1 PAK transcription inhibitor, a Group 1 PAK clearance agent, an agent that binds a Group 1 PAK to prevent its interaction with one or more cellular proteins, and a Group 1 antagonist. The subject is an animal or human, and is preferably a mammal, preferably human.

In other methods PAK inhibitors, binding molecules, and clearance agents provided herein are administered to a human subject with a neuropsychiatric condition to increasing dendritic spine density in the subject. The method includes: administering to the human subject a pharmacological composition comprising a therapeutically effective amount of at least one of the compounds chosen from the group consisting of: a PAK transcription inhibitor, a PAK clearance agent, an agent that binds PAK to prevent its interaction with one or more cellular or extracellular proteins, and a PAK antagonist. In some preferred embodiments, the pharmacological composition comprises a therapeutically effective amount of at least one of the compounds chosen from the group consisting of: a Group 1 PAK transcription inhibitor, a Group 1 PAK clearance agent, an agent that binds a Group 1 PAK to prevent its interaction with one or more cellular proteins, and a Group 1 PAK antagonist. The subject is an animal, and is preferably a mammal, preferably human.

In other methods PAK inhibitors, binding molecules, and clearance agents provided herein are administered to a human subject with a neuropsychiatric condition to reverse some or all defects in dendritic spine morphology, spine size and/or spine plasticity in the subject. The method includes: administering to the human subject a pharmacological composition comprising a therapeutically effective amount of at least one of the compounds chosen from the group consisting of: a PAK transcription inhibitor, a PAK clearance agent, an agent that binds PAK to prevent its interaction with one or more cellular or extracellular proteins, and a PAK antagonist. In some preferred embodiments, the pharmacological composition comprises a therapeutically effective amount of at least one of the compounds chosen from the group consisting of: a Group 1 PAK transcription inhibitor, a Group 1 PAK clearance agent, an agent that binds a Group 1 PAK to prevent its interaction with one or more cellular proteins, and a Group 1 PAK antagonist. The subject is an animal, and is preferably a mammal, preferably human.

In other methods PAK inhibitors, binding molecules, and clearance agents provided herein are administered to a human subject with a neuropsychiatric condition to decrease one or more disease symptoms or pathologies in the subject. A disease symptom or pathology can be, as nonlimiting examples, the presence, size, or amount of intracellular or extracellular aggregates or malformations, cell death, ataxia, tremors, seizures, cognitive decline, or psychosis. The method includes: administering to the human subject a pharmacological composition comprising a therapeutically effective amount of at least one of the compounds chosen from the group consisting of: a PAK transcription inhibitor, a PAK clearance agent, an agent that binds PAK to prevent its interaction with one or more cellular or extracellular proteins, and a PAK antagonist. In some preferred embodiments, the pharmacological composition comprises a therapeutically effective amount of at least one of the compounds chosen from the group consisting of: a Group 1 PAK transcription inhibitor, a Group 1 PAK clearance agent, an agent that binds a Group 1 PAK to prevent its interaction with one or more cellular proteins, and a Group 1 PAK antagonist. The subject is an animal or human, and is preferably a mammal, preferably human.

### Examples of Pharmaceutical Compositions and Methods of Administration

Pharmaceutical compositions are formulated using one or more physiologically acceptable carriers including excipients and auxiliaries which facilitate processing of the active compounds into preparations which are used pharmaceutically. Proper formulation is dependent upon the route of administration chosen. A summary of pharmaceutical compositions is found, for example, in Remington: The Science and Practice of Pharmacy, Nineteenth Ed (Ea hston, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

Provided herein are pharmaceutical compositions that include one or more PAK inhibitors and a pharmaceutically acceptable diluent(s), excipient(s), or carrier(s). In addition, a PAK inhibitor is optionally administered as pharmaceutical compositions in which it is mixed with other active ingredients, as in combination therapy. In some embodiments, the pharmaceutical compositions includes other medicinal or pharmaceutical agents, carriers, adjuvants, such as preserving, stabilizing, wetting or emulsifying agents, solution promoters, salts for regulating the osmotic pressure, and/or buffers. In addition, the pharmaceutical compositions also contain other therapeutically valuable substances.

A pharmaceutical composition, as used herein, refers to a mixture of a PAK inhibitor with other chemical components, such as carriers, stabilizers, diluents, dispersing agents, suspending agents, thickening agents, and/or excipients. The pharmaceutical composition facilitates administration of a PAK inhibitor to an organism. In practicing the methods of treatment or use provided herein, therapeutically effective amounts of a PAK inhibitor are administered in a pharmaceutical composition to a mammal having a condition, disease, or disorder to be treated. Preferably, the mammal is a human. A therapeutically effective amount varies depending on the severity and stage of the condition, the age and relative health of the subject, the potency of a PAK inhibitor used and other factors. The PAK inhibitor is optionally used singly or in combination with one or more therapeutic agents as components of mixtures.

The pharmaceutical formulations described herein are optionally administered to a subject by multiple administration mutes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

A PAK inhibitor is optionally formulated for delivery across the blood-brain barrier. In some embodiments, provided herein is a pharmaceutical composition comprising a PAK inhibitor and an agent that facilitates the transport of the PAK inhibitor across the blood brain barrier. In certain embodiments, an agent that facilitates the transport of a PAK inhibitor is covalently attached to a PAK inhibitor. In some instances, PAK inhibitors described herein are modified by covalent attachment to a lipophilic carrier or co-formulation with a lipophilic carrier. In some embodiments, a PAK inhibitor is covalently attached to a lipophilic carrier, such as e.g., DHA, or a fatty acid. In some embodiments, a PAK inhibitor is covalently attached to artificial low density lipoprotein particles. In some instances, carrier systems facilitate the passage of PAK inhibitors described herein across the blood-brain bather and include but are not limited to, the use of a dihydropyridine pyridinium salt carrier redox system for delivery of drug species across the blood brain barrier. In some instances a PAK inhibitor described herein is coupled to a lipophilic phosphonate derivative. In certain instances, PAK inhibitors described herein are conjugated to PEG-oligomers/polymers or aprotinin derivatives and analogs. In some instances, an increase in influx of a PAK inhibitor described herein across the blood brain barrier is achieved by modifying a PAK inhibitor described herein (e.g., by reducing or increasing the number of charged groups on the compound) and enhancing affinity for a blood brain barrier transporter. In certain instances, a PAK inhibitor is co-administered with an agent that reduces or inhibits efflux across the blood brain barrier, e.g. an inhibitor of P-glycoprotein pump (PGP) mediated efflux (e.g., cyclosporin, SCH66336 (lonafarnib, Schering)).

The pharmaceutical compositions will include at least one PAK inhibitor, as an active ingredient in free-acid or free-base form, or in a pharmaceutically acceptable salt form. In addition, the methods and pharmaceutical compositions described herein include the use of N-oxides, crystalline forms (also known as polymorphs), as well as active metabolites of these PAK inhibitors having the same type of activity. In some situations, PAK inhibitors exist as tautomers. All tautomers are included within the scope of the compounds presented herein. Additionally, a PAK inhibitor exists in unsolvated as well as solvated forms with pharmaceutically acceptable solvents such as water, ethanol, and the like. The solvated forms of a PAK inhibitors presented herein are also considered to be disclosed herein.

“Carrier materials” include any commonly used excipients in pharmaceutics and should be selected on the basis of compatibility with compounds disclosed herein, such as, a PAK inhibitor, and the release profile properties of the desired dosage form. Exemplary carrier materials include, e.g., binders, suspending agents, disintegration agents, filling agents, surfactants, solubilizers, stabilizers, lubricants, wetting agents, diluents, and the like.

Moreover, the pharmaceutical compositions described herein, which include a PAK inhibitor, are formulated into any suitable dosage form, including but not limited to, aqueous oral dispersions, liquids, gels, syrups, elixirs, slurries, suspensions and the like, for oral ingestion by a patient to be treated, solid oral dosage forms, aerosols, controlled release formulations, fast melt formulations, effervescent formulations, lyophilized formulations, tablets, powders, pills, dragees, capsules, delayed release formulations, extended release formulations, pulsatile release formulations, multiparticulate formulations, and mixed immediate release and controlled release formulations.

Pharmaceutical preparations for oral use are optionally obtained by mixing one or more solid excipient with a PAK inhibitor, optionally grinding the resulting mixture, and processing the mixture of granules, after adding suitable auxiliaries, if desired, to obtain tablets or dragee cores. Suitable excipients include, for example, fillers such as sugars, including lactose, sucrose, mannitol, or sorbitol; cellulose preparations such as, for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. If desired, disintegrating agents are added, such as the cross linked croscarmellose sodium, polyvinylpyrrolidone, agar, or alginic acid or a salt thereof such as sodium alginate.

Dragee cores are provided with suitable coatings. For this purpose, concentrated sugar solutions are generally used, which optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments are optionally added to the tablets or dragee coatings for identification or to characterize different combinations of active compound doses.

In some embodiments, the solid dosage forms disclosed herein are in the form of a tablet, (including a suspension tablet, a fast-melt tablet, a bite-disintegration tablet, a rapid-disintegration tablet, an effervescent tablet, or a caplet), a pill, a powder (including a sterile packaged powder, a dispensable powder, or an effervescent powder) a capsule (including both soft or hard capsules, e.g., capsules made from animal-derived gelatin or plant-derived HPMC, or “sprinkle capsules”), solid dispersion, solid solution, bioerodible dosage form, controlled release formulations, pulsatile release dosage forms, multiparticulate dosage forms, pellets, granules, or an aerosol. In other embodiments, the pharmaceutical formulation is in the form of a powder. In still other embodiments, the pharmaceutical formulation is in the form of a tablet, including but not limited to, a fast-melt tablet. Additionally, pharmaceutical formulations of a PAK inhibitor are optionally administered as a single capsule or in multiple capsule dosage form. In some embodiments, the pharmaceutical formulation is administered in two, or three, or four, capsules or tablets.

In another aspect, dosage forms include microencapsulated formulations. In some embodiments, one or more other compatible materials are present in the microencapsulation material. Exemplary materials include, but are not limited to, pH modifiers, erosion facilitators, anti-foaming agents, antioxidants, flavoring agents, and carrier materials such as binders, suspending agents, disintegration agents, filling agents, surfactants, solubilizers, stabilizers, lubricants, wetting agents, and diluents.

Exemplary microencapsulation materials useful for delaying the release of the formulations including a PAK inhibitor, include, but are not limited to, hydroxypropyl cellulose ethers (HPC) such as Klucel® or Nisso HPC, low-substituted hydroxypropyl cellulose ethers (L-HPC), hydroxypropyl methyl cellulose ethers (HPMC) such as Seppifilm-LC, Pharmacoat®, Metolose SR, Methocel®-E, Opadry YS, PrimaFlo, Benecel MP824, and Benecel MP843, methylcellulose polymers such as Methocel®-A, hydroxypropylmethylcellulose acetate stearate Aqoat (HF-LS, HF-LG, HF-MS) and Metolose®, Ethylcelluloses (EC) and mixtures thereof such as E461, Ethocel®, Aqualon®-EC, Surelease®, Polyvinyl alcohol (PVA) such as Opadry AMB, hydroxyethylcelluloses such as Natrosol®, carboxymethylcelluloses and salts of carboxymethylcelluloses (CMC) such as Aqualon®-CMC, polyvinyl alcohol and polyethylene glycol co-polymers such as Kollicoat IR®, monoglycerides (Myverol), triglycerides (KLX), polyethylene glycols, modified food starch, acrylic polymers and mixtures of acrylic polymers with cellulose ethers such as Eudragit® EPO, Eudragit® L30D-55, Eudragit® FS 30D Eudragit® L100-55, Eudragit® L100, Eudragit® S100, Eudragit® RD100, Eudragit® E100, Eudragit® L12.5, Eudragit® S12.5, Eudragit® NE30D, and Eudragit® NE 40D, cellulose acetate phthalate, sepifilms such as mixtures of HPMC and stearic acid, cyclodextrins, and mixtures of these materials.

The pharmaceutical solid oral dosage forms including formulations described herein, which include a PAK inhibitor, are optionally further formulated to provide a controlled release of a PAK inhibitor. Controlled release refers to the release of a PAK inhibitor from a dosage form in which it is incorporated according to a desired profile over an extended period of time. Controlled release profiles include, for example, sustained release, prolonged release, pulsatile release, and delayed release profiles. In contrast to immediate release compositions, controlled release compositions allow delivery of an agent to a subject over an extended period of time according to a predetermined profile. Such release rates provide therapeutically effective levels of agent for an extended period of time and thereby provide a longer period of pharmacologic response while minimizing side effects as compared to conventional rapid release dosage forms. Such longer periods of response provide for many inherent benefits that are not achieved with the corresponding short acting, immediate release preparations.

In other embodiments, the formulations described herein, which include a PAK inhibitor, are delivered using a pulsatile dosage form. A pulsatile dosage form is capable of providing one or more immediate release pulses at predetermined time points after a controlled lag time or at specific sites. Pulsatile dosage forms including the formulations described herein, which include a PAK inhibitor, are optionally administered using a variety of pulsatile formulations that include, but are not limited to, those described in U.S. Pat. Nos. 5,011,692, 5,017,381, 5,229,135, and 5,840,329. Other pulsatile release dosage forms suitable for use with the present formulations include, but are not limited to, for example, U.S. Pat. Nos. 4,871,549, 5,260,068, 5,260,069, 5,508,040, 5,567,441 and 5,837,284.

Liquid formulation dosage forms for oral administration are optionally aqueous suspensions selected from the group including, but not limited to, pharmaceutically acceptable aqueous oral dispersions, emulsions, solutions, elixirs, gels, and syrups. See, e.g., Singh et al., Encyclopedia of Pharmaceutical Technology, 2nd Ed., pp. 754-757 (2002). In addition to a PAK inhibitor, the liquid dosage forms optionally include additives, such as: (a) disintegrating agents; (b) dispersing agents; (c) wetting agents; (d) at least one preservative, (e) viscosity enhancing agents, (f) at least one sweetening agent, and (g) at least one flavoring agent. In some embodiments, the aqueous dispersions further includes a crystal-forming inhibitor.

In some embodiments, the pharmaceutical formulations described herein are elf-emulsifying drug delivery systems (SEDDS). Emulsions are dispersions of one immiscible phase in another, usually in the form of droplets. Generally, emulsions are created by vigorous mechanical dispersion. SEDDS, as opposed to emulsions or microemulsions, spontaneously form emulsions when added to an excess of water without any external mechanical dispersion or agitation. An advantage of SEDDS is that only gentle mixing is required to distribute the droplets throughout the solution. Additionally, water or the aqueous phase is optionally added just prior to administration, which ensures stability of an unstable or hydrophobic active ingredient. Thus, the SEDDS provides an effective delivery system for oral and parenteral delivery of hydrophobic active ingredients. In some embodiments, SEDDS provides improvements in the bioavailability of hydrophobic active ingredients. Methods of producing self-emulsifying dosage forms include, but are not limited to, for example, U.S. Pat. Nos. 5,858,401, 6,667,048, and 6,960,563.

Suitable intranasal formulations include those described in, for example, U.S. Pat. Nos. 4,476,116, 5,116,817 and 6,391,452. Nasal dosage forms generally contain large amounts of water in addition to the active ingredient Minor amounts of other ingredients such as pH adjusters, emulsifiers or dispersing agents, preservatives, surfactants, gelling agents, or buffering and other stabilizing and solubilizing agents are optionally present

For administration by inhalation, a PAK inhibitor is optionally in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix of a PAK inhibitor and a suitable powder base such as lactose or starch.

Buccal formulations that include a PAK inhibitor include, but are not limited to, U.S. Pat. Nos. 4,229,447, 4,596,795, 4,755,386, and 5,739,136. In addition, the buccal dosage forms described herein optionally further include a bioerodible (hydrolysable) polymeric carrier that also serves to adhere the dosage form to the buccal mucosa. The buccal dosage form is fabricated so as to erode gradually over a predetermined time period, wherein the delivery of a PAK inhibitor, is provided essentially throughout. Buccal drug delivery avoids the disadvantages encountered with oral drug administration, e.g., slow absorption, degradation of the active agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which may be obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the compositions optionally take the form of tablets, lozenges, or gels formulated in a conventional manner.

Transdermal formulations of a PAK inhibitor are administered for example by those described in U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal formulations described herein include at least three components: (1) a formulation of a PAK inhibitor; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

In some embodiments, formulations suitable for transdermal administration of a PAK inhibitor employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery of a PAK inhibitor is optionally accomplished by means of iontophoretic patches and the like. Additionally, transdermal patches provide controlled delivery of a PAK inhibitor. The rate of absorption is optionally slowed by using rate-controlling membranes or by trapping a PAK inhibitor within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing a PAK inhibitor optionally with carriers, optionally a rate controlling barrier to deliver a PAK inhibitor to the skin of the host at a controlled and predetermined rate over a prolonged period of time, and means to secure the device to the skin.

Formulations that include a PAK inhibitor suitable for intramuscular, subcutaneous, or intravenous injection include physiologically acceptable sterile aqueous or non-aqueous solutions, dispersions, suspensions or emulsions, and sterile powders for reconstitution into sterile injectable solutions or dispersions. Examples of suitable aqueous and non-aqueous carriers, diluents, solvents, or vehicles including water, ethanol, polyols (propyleneglycol, polyethylene-glycol, glycerol, cremophor and the like), suitable mixtures thereof, vegetable oils (such as olive oil) and injectable organic esters such as ethyl oleate. Proper fluidity is maintained, for example, by the use of a coating such as lecithin, by the maintenance of the required particle size in the case of dispersions, and by the use of surfactants. Formulations suitable for subcutaneous injection also contain optional additives such as preserving, wetting, emulsifying, and dispensing agents.

For intravenous injections, a PAK inhibitor is optionally formulated in aqueous solutions, preferably in physiologically compatible buffers such as Hank's solution, Ringer's solution, or physiological saline buffer. For transmucosal administration, penetrants appropriate to the bather to be permeated are used in the formulation. For other parenteral injections, appropriate formulations include aqueous or nonaqueous solutions, preferably with physiologically compatible buffers or excipients.

Parenteral injections optionally involve bolus injection or continuous infusion. Formulations for injection are optionally presented in unit dosage form, e.g., in ampoules or in multi dose containers, with an added preservative. In some embodiments, the pharmaceutical composition described herein are in a form suitable for parenteral injection as a sterile suspensions, solutions or emulsions in oily or aqueous vehicles, and contain formulatory agents such as suspending, stabilizing and/or dispersing agents. Pharmaceutical formulations for parenteral administration include aqueous solutions of a PAK inhibitor in water soluble form. Additionally, suspensions of a PAK inhibitor are optionally prepared as appropriate oily injection suspensions.

In some embodiments, a PAK inhibitor is administered topically and formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

The PAK inhibitor is also optionally formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

### Examples of Methods of Dosing and Treatment Regimens

The PAK inhibitor is optionally used in the preparation of medicaments for the prophylactic and/or therapeutic treatment of neuropsychiatric diseases or conditions that would benefit, at least in part, from amelioratoin. In addition, a method for treating any of the diseases or conditions described herein in a subject in need of such treatment, involves administration of pharmaceutical compositions containing at least one PAK inhibitor described herein, or a pharmaceutically acceptable salt, pharmaceutically acceptable N-oxide, pharmaceutically active metabolite, pharmaceutically acceptable prodrug, or pharmaceutically acceptable solvate thereof, in therapeutically effective amounts to said subject.

In the case wherein the patient's condition does not improve, upon the doctor's discretion the administration of a PAK inhibitor is optionally administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of a PAK inhibitor is optionally given continuously; alternatively, the dose of drug being administered is temporarily reduced or temporarily suspended for a certain length of time (i.e., a “drug holiday”). The length of the drug holiday optionally varies between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, or 365 days. The dose reduction during a drug holiday includes from 10%-100%, including, by way of example only, 10%, 15%, 20%, 25%, 30%, 35%, 40%, 45%, 50%, 55%, 60%, 65%, 70%, 75%, 80%, 85%, 90%, 95%, or 100%.

Once improvement of the patient's conditions has occurred, a maintenance dose is administered if necessary. Subsequently, the dosage or the frequency of administration, or both, is reduced, as a function of the symptoms, to a level at which the improved disease, disorder or condition is retained. In some embodiments, patients require intermittent treatment on a long-term basis upon any recurrence of symptoms.

In some embodiments, the pharmaceutical compositions described herein are in unit dosage forms suitable for single administration of precise dosages. In unit dosage form, the formulation is divided into unit doses containing appropriate quantities of one or more PAK inhibitor. In some embodiments, the unit dosage is in the form of a package containing discrete quantities of the formulation. Non-limiting examples are packaged tablets or capsules, and powders in vials or ampoules. In some embodiments, aqueous suspension compositions are packaged in single-dose non-reclosable containers. Alternatively, multiple-dose reclosable containers are used, in which case it is typical to include a preservative in the composition. By way of example only, formulations for parenteral injection are presented in unit dosage form, which include, but are not limited to ampoules, or in multi dose containers, with an added preservative.

The daily dosages appropriate for a PAK inhibitor are from about 0.01 to 2.5 mg/kg per body weight. An indicated daily dosage in the larger mammal, including, but not limited to, humans, is in the range from about 0.5 mg to about 100 mg, conveniently administered in divided doses, including, but not limited to, up to four times a day or in extended release form. Suitable unit dosage forms for oral administration include from about 1 to 50 mg active ingredient. The foregoing ranges are merely suggestive, as the number of variables in regard to an individual treatment regime is large, and considerable excursions from these recommended values are not uncommon. Such dosages are optionally altered depending on a number of variables, not limited to the activity of a PAK inhibitor used, the disease or condition to be treated, the mode of administration, the requirements of the individual subject, the severity of the disease or condition being treated, and the judgment of the practitioner.

Toxicity and therapeutic efficacy of such therapeutic regimens are optionally determined in cell cultures or experimental animals, including, but not limited to, the determination of the LD50 (the dose lethal to 50% of the population) and the ED50 (the dose therapeutically effective in 50% of the population). The dose ratio between the toxic and therapeutic effects is the therapeutic index, which is expressed as the ratio between LD50 and ED50. PAK inhibitors exhibiting high therapeutic indices are preferred. The data obtained from cell culture assays and animal studies is optionally used in formulating a range of dosage for use in human. The dosage of such PAK inhibitors lies preferably within a range of circulating concentrations that include the ED50 with minimal toxicity. The dosage optionally varies within this range depending upon the dosage form employed and the route of administration utilized.

### Combination Treatments

The PAK inhibitor compositions described herein are also optionally used in combination with other therapeutic reagents that are selected for their therapeutic value for the condition to be treated. In general, the compositions described herein and, in embodiments where combinational therapy is employed, other agents do not have to be administered in the same pharmaceutical composition, and, because of different physical and chemical characteristics, are optionally administered by different routes. The initial administration is generally made according to established protocols, and then, based upon the observed effects, the dosage, modes of administration and times of administration subsequently modified.

In certain instances, it is appropriate to administer at least one PAK inhibitor composition described herein in combination with another therapeutic agent. By way of example only, if one of the side effects experienced by a patient upon receiving one of a PAK inhibitor compositions described herein is nausea, then it is appropriate to administer an anti-nausea agent in combination with the initial therapeutic agent. Or, by way of example only, the therapeutic effectiveness of a PAK inhibitor is enhanced by administration of an adjuvant (i.e., by itself the adjuvant has minimal therapeutic benefit, but in combination with another therapeutic agent, the overall therapeutic benefit to the patient is enhanced). Or, by way of example only, the benefit experienced by a patient is increased by administering a PAK inhibitor with another therapeutic agent (which also includes a therapeutic regimen) that also has therapeutic benefit. In any case, regardless of the disease, disorder or condition being treated, the overall benefit experienced by the patient is either simply additive of the two therapeutic agents or the patient experiences a synergistic benefit.

Therapeutically-effective dosages vary when the drugs are used in treatment combinations. Methods for experimentally determining therapeutically-effective dosages of drugs and other agents for use in combination treatment regimens are documented methodologies. One example of such a method is the use of metronomic dosing, i.e., providing more frequent, lower doses in order to minimize toxic side effects. Combination treatment further includes periodic treatments that start and stop at various times to assist with the clinical management of the patient.

In any case, the multiple therapeutic agents (one of which is a PAK inhibitor described herein) is administered in any order, or even simultaneously. If simultaneously, the multiple therapeutic agents are optionally provided in a single, unified form, or in multiple forms (by way of example only, either as a single pill or as two separate pills). In some embodiments, one of the therapeutic agents is given in multiple doses, or both are given as multiple doses. If not simultaneous, the timing between the multiple doses optionally varies from more than zero weeks to less than four weeks. In addition, the combination methods, compositions and formulations are not to be limited to the use of only two agents; the use of multiple therapeutic combinations are also envisioned.

It is understood that the dosage regimen to treat, prevent, or ameliorate the condition(s) for which relief is sought, is optionally modified in accordance with a variety of factors. These factors include the disorder from which the subject suffers, as well as the age, weight, sex, diet, and medical condition of the subject. Thus, the dosage regimen actually employed varies widely, in some embodiments, and therefore deviates from the dosage regimens set forth herein.

The pharmaceutical agents which make up the combination therapy disclosed herein are optionally a combined dosage form or in separate dosage forms intended for substantially simultaneous administration. The pharmaceutical agents that make up the combination therapy are optionally also be administered sequentially, with either therapeutic compound being administered by a regimen calling for two-step administration. The two-step administration regimen optionally calls for sequential administration of the active agents or spaced-apart administration of the separate active agents. The time period between the multiple administration steps ranges from, a few minutes to several hours, depending upon the properties of each pharmaceutical agent, such as potency, solubility, bioavailability, plasma half-life and kinetic profile of the pharmaceutical agent. Circadian variation of the target molecule concentration are optionally used to determine the optimal dose interval.

In addition, a PAK inhibitor is optionally used in combination with procedures that provide additional or synergistic benefit to the patient. By way of example only, patients are expected to find therapeutic and/or prophylactic benefit in the methods described herein, wherein pharmaceutical composition of a PAK inhibitor and/or combinations with other therapeutics are combined with genetic testing to determine whether that individual is a carrier of a mutant gene that is correlated with certain diseases or conditions.

A PAK inhibitor and the additional therapy(ies) are optionally administered before, during or after the occurrence of a disease or condition, and the timing of administering the composition containing a PAK inhibitor varies in some embodiments. Thus, for example, a PAK inhibitor is used as a prophylactic and are administered continuously to subjects with a propensity to develop conditions or diseases in order to prevent the occurrence of the disease or condition. The PAK inhibitors and compositions are optionally administered to a subject during or as soon as possible after the onset of the symptoms. The administration of the compounds are optionally initiated within the first 48 hours of the onset of the symptoms, preferably within the first 48 hours of the onset of the symptoms, more preferably within the first 6 hours of the onset of the symptoms, and most preferably within 3 hours of the onset of the symptoms. The initial administration is optionally via any route practical, such as, for example, an intravenous injection, a bolus injection, infusion over 5 minutes to about 5 hours, a pill, a capsule, transdermal patch, buccal delivery, and the like, or combination thereof. A PAK inhibitor is preferably administered as soon as is practicable after the onset of a disease or condition is detected or suspected, and for a length of time necessary for the treatment of the disease, such as, for example, from about 1 month to about 3 months. The length of treatment optionally varies for each subject, and the length is then determined using the known criteria. For example, a PAK inhibitor or a formulation containing a PAK inhibitor is administered for at least 2 weeks, preferably about 1 month to about 5 years, and more preferably from about 1 month to about 3 years.

Exemplary Therapeutic Agents for Use in Combination with a PAK Inhibitor Composition

In certain instances, provided herein are compositions and/or therapies comprising a therapeutically effective amount of any compound described herein and an additional therapeutic agent. In some instances the additional therapeutic agent is an agent for treating a psychotic disorder, a mood disorder, epilepsy, Huntington's disease, Parkinson's disease, or a modulator of an upstream regulator of PAKs (e.g., PDK1 inhibitors, PI3 kinase inhibitors, CDK5 inhibitors, GRB2 inhibitors, NCK inhibitors, ETK inhibitors, Rac inhibitors, Cdc42 inhibitors). In some instances, the additional therapeutic agent is a blood brain barrier facilitator.

### Agents for Treating Psychotic Disorders

Where a subject is suffering from or at risk of suffering from a psychotic disorder (e.g., schizophrenia), a PAK inhibitor composition described herein is optionally used together with one or more agents or methods for treating a psychotic disorder in any combination. Alternatively, a PAK inhibitor composition described herein is administered to a patient who has been prescribed an agent for treating a psychotic disorder. Alternatively, a PAK inhibitor composition described herein is administered to a patient who is refractory to or being unsatisfactorily treated with an agent for treating a psychotic disorder.

Examples of therapeutic agents/treatments for treating a psychotic disorder include, but are not limited to, any of the following: typical antipsychotics, e.g., Chlorpromazine (Largactil, Thorazine), Fluphenazine (Prolixin), Haloperidol (Haldol, Serenace), Molindone, Thiothixene (Navane), Thioridazine (Mellaril), Trifluoperazine (Stelazine), Loxapine, Perphenazine, Prochlorperazine (Compazine, Buccastem, Stemetil), Pimozide (Orap), Zuclopenthixol; and atypical antipsychotics, e.g., LY2140023, Clozapine, Risperidone, Olanzapine, Quetiapine, Ziprasidone, Aripiprazole, Paliperidone, Asenapine, Iloperidone, Sertindole, Zotepine, Amisulpride, Bifeprunox, and Melperone.

### Agents for Treating Mood Disorders

Where a subject is suffering from or at risk of suffering from a mood disorder (e.g., clinical clinical depression), a PAK inhibitor composition described herein is optionally used together with one or more agents or methods for treating a mood disorder in any combination. Alternatively, a PAK inhibitor composition described herein is administered to a patient who has been prescribed an agent for treating a mood disorder. Alternatively, a PAK inhibitor composition described herein is administered to a patient who is refractory to or being unsatisfactorily treated with an agent for treating a mood disorder.

Examples of therapeutic agents/treatments for treating a mood disorder include, but are not limited to, any of the following: selective serotonin reuptake inhibitors (SSRIs) such as citalopram (Celexa), escitalopram (Lexapro, Esipram), fluoxetine (Prozac), paroxetine (Paxil, Seroxat), sertraline (Zoloft), fluvoxamine (Luvox); serotonin-norepinephrine reuptake inhibitors (SNRIs) such as venlafaxine (Effexor), desvenlafaxine, nefazodone, milnacipran, duloxetine (Cymbalta), bicifadine; tricyclic antidepressants such as amitriptyline, amoxapine, butriptyline, clomipramine, desipramine, dosulepin, doxepin, impramine, lofepramine, nortriptyline; monoamine oxidase inhibitors (MAOIs) such as isocarboxazid, linezolid, moclobemide, nialamide, phenelzine, selegiline, tranylcypromine, trimipramine; and other agents such as mirtazapine, reboxetine, viloxazine, malprotiline, and bupropion.

### Agents for Treating Epilepsy

Where a subject is suffering from or at risk of suffering from epilepsy, a PAK inhibitor composition described herein is optionally used together with one or more agents or methods for treating epilepsy in any combination. Alternatively, a PAK inhibitor composition described herein is administered to a patient who has been prescribed an agent for treating epilepsy. Alternatively, a PAK inhibitor composition described herein is administered to a patient who is refractory to or being unsatisfactorily treated with an agent for treating epilepsy.

Examples of therapeutic agents/treatments for treating epilepsy include, but are not limited to, any of the following: carbamazepine, clobazam, clonazepam, ethosuximide, felbamate, fosphenyloin, gabapentin, lamotrigine, levetiracetam, oxcarbazepine, phenobarbital, phenyloin, pregabalin, primidone, sodium valproate, tiagabine, topiramate, valproate semisodium, valproic acid, vigabatrin, and zonisamide.

### Agents for Treating Huntington's Disease

Where a subject is suffering from or at risk of suffering from Huntingtin's disease, a PAK inhibitor composition described herein is optionally used together with one or more agents or methods for treating Huntingtin's disease in any combination. Alternatively, a PAK inhibitor composition described herein is administered to a patient who has been prescribed an agent for treating Huntington's disease. Alternatively, a PAK inhibitor composition described herein is administered to a patient who is refractory to or being unsatisfactorily treated with an agent for treating Huntington's disease.

Examples of therapeutic agents/treatments for treating Huntingtin's disease include, but are not limited to, any of the following: omega-3 fatty acids, miraxion, Haloperidol, dopamine receptor blockers, creatine, cystamine, cysteamine, clonazepam, clozapine, Coenzyme Q10, minocycline, antioxidants, antidepressants (notably, but not exclusively, selective serotonin reuptake inhibitors SSRIs, such as sertraline, fluoxetine, and paroxetine), select dopamine antagonists, such as tetrabenazine; and RNAi knockdown of mutant huntingtin (mHtt).

### Agents for Treating Parkinson's Disease

Where a subject is suffering from or at risk of suffering from Parkinson's Disease, a PAK inhibitor composition described herein is optionally used together with one or more agents or methods for treating Parkinson's disease in any combination. Alternatively, a PAK inhibitor composition described herein is administered to a patient who has been prescribed an agent for treating Parkinson's disease. Alternatively, a PAK inhibitor composition described herein is administered to a patient who is refractory to or being unsatisfactorily treated with an agent for treating Parkinson's disease.

Examples of therapeutic agents/treatments for treating Parkinson's Disease include, but are not limited to any of the following: L-dopa, carbidopa, benserazide, tolcapone, entacapone, bromocriptine, pergolide, pramipexole, ropinirole, cabergoline, apomorphine, lisuride, selegiline, or rasagiline

### Modulators of Upstream Regulators of PAKs

In some embodiments, a modulator of an upstream regulator of PAKs is an indirect inhibitor of PAK. In certain instances, a modulator of an upstream regulator of PAKs is a modulator of PDK1. In some instances, a modulator of PDK1 reduces of inhibits the activity of PDK1. In some instances a PDK1 inhibitor is an antisense compound (e.g., any PDK1 inhibitor described in U.S. Pat. No. 6,124,272, which PDK1 inhibitor is incorporated herein by reference). In some instances, a PDK1 inhibitor is a compound described in e.g., U.S. Pat. Nos. 7,344,870, and 7,041,687, which PDK1 inhibitors are incorporated herein by reference. In some embodiments, an indirect inhibitor of PAK is a modulator of a PI3 kinase. In some instances a modulator of a P13 kinase is a PI3 kinase inhibitor. In some instances, a PI3 kinase inhibitor is an antisense compound (e.g., any PI3 kinase inhibitor described in WO 2001/018023, which PI3 kinase inhibitors are incorporated herein by reference). In some instances, an inhibitor of a PI3 kinase is 3-morpholino-5-phenylnaphthalen-1(4H)-one (LY294002), or a peptide based covalent conjugate of LY294002, (e.g., SF1126, Semaphore pharmaceuticals). In certain embodiments, an indirect inhibitor of PAK is a modulator of Cdc42. In certain embodiments, a modulator of Cdc42 is an inhibitor of Cdc42. In certain embodiments, a Cdc42 inhibitor is an antisense compound (e.g., any Cdc42 inhibitor described in U.S. Pat. No. 6,410,323, which Cdc42 inhibitors are incorporated herein by reference). In some instances, an indirect inhibitor of PAK is a modulator of GRB2. In some instances, a modulator of GRB2 is an inhibitor of GRB2. In some instances a GRB2 inhibitor is a GRb2 inhibitor described in e.g., U.S. Pat. No. 7,229,960, which GRB2 inhibitor is incorporated by reference herein. In certain embodiments, an indirect inhibitor of PAK is a modulator of NCK. In certain embodiments, an indirect inhibitor of PAK is a modulator of ETK. In some instances, a modulator of ETK is an inhibitor of ETK. In some instances an ETK inhibitor is a compound e.g., [symbol] -Cyano-(3,5-di-t-butyl-4-hydroxy)thiocinnamide (AG 879).

### Blood Brain Barrier Facilitators

In some instances, a PAK inhibitor is optionally administered in combination with a blood brain bather facilitator. In certain embodiments, an agent that facilitates the transport of a PAK inhibitor is covalently attached to the PAK inhibitor. In some instances, PAK inhibitors described herein are modified by covalent attachment to a lipophilic carrier or co-formulation with a lipophilic carrier. In some embodiments, a PAK inhibitor is covalently attached to a lipophilic carrier, such as e.g., DHA, or a fatty acid. In some embodiments, a PAK inhibitor is covalently attached to artificial low density lipoprotein particles. In some instances, carrier systems facilitate the passage of PAK inhibitors described herein across the blood-brain barrier and include but are not limited to, the use of a dihydropyridine pyridinium salt carrier redox system for delivery of drug species across the blood brain bather. In some instances a PAK inhibitor described herein is coupled to a lipophilic phosphonate derivative. In certain instances, PAK inhibitors described herein are conjugated to PEG-oligomers/polymers or aprotinin derivatives and analogs. In some instances, an increase in influx of a PAK inhibitor described herein across the blood brain bather is achieved by modifying A PAK inhibitor described herein (e.g., by reducing or increasing the number of charged groups on the compound) and enhancing affinity for a blood brain barrier transporter. In certain instances, a PAK inhibitor is co-administered with an agent that reduces or inhibits efflux across the blood brain barrier, e.g. an inhibitor of P-glycoprotein pump (PGP) mediated efflux (e.g., cyclosporin, SCH66336 (lonafarnib, Schering)).

In some instances, a PAK inhibitor polypeptide is delivered to one or more brain regions of a individual by administration of a viral expression vector, e.g., an AAV vector, a lentiviral vector, an adenoviral vector, or a HSV vector. A number of viral vectors for delivery of therapeutic proteins are described in, e.g., U.S. Pat. Nos., 7,244,423, 6,780,409, 5,661,033. In some embodiments, the PAK inhibitor polypeptide to be expressed is under the control of an inducible promoter (e.g., a promoter containing a tet-operator). Inducible viral expression vectors include, for example, those described in U.S. Pat. No. 6,953,575. Inducible expression of a PAK inhibitor polypeptide allows for tightly controlled and reversible increases of PAK inhibitor polypeptide expression by varying the dose of an inducing agent (e.g., tetracycline) administered to an individual.

Group I mGluR Antagonists

Reduction of signaling from a Group I mGluR (mGluR5) in vivo by genetic engineering (using mGluR5 knock-out heterozygote animals) leads to a reversal of the dendritic spine and behavioral defects observed in an animal model of Fragile X syndrome. Where a subject is suffering from or at risk of suffering from Fragile X syndrome, autism, mental retardation, Down's syndrome, or a NC described herein (e.g., schizophrenia), a PAK inhibitor composition described herein is optionally used together with one or Group I mGluR antagonists. Group I mGluR antagonists include antagonists that are mGluR1-selective antagonists, mGluR5-selective antagonists, or antagonists that antagonize both mGluR1 and mGluR5. In some embodiments, a PAK inhibitor composition is used in combination with an mGluR5-selective antagonist. In some embodiments, a PAK inhibitor composition is used in combination with an mGluR1 selective antagonist. In some embodiments, a PAK inhibitor composition is used in combination with a Group I mGluR antagonist that antagonizes both mGluR1 and mGluR5 (i.e., an antagonist that is not selective for mGluR1 or mGluR5). As used herein, the term “selective antagonist” indicates that the antagonist has an ED₅₀ for antagonizing a first receptor (e.g., mGluR5) that is at least about 10 fold to about 1000 fold lower, e.g., 11, 20, 30, 40, 50, 100, 105, 125, 135, 150, 200, 300, 400, 500, 600, 700, 800, 900, or any other fold lower from about 10 fold to about 1000 fold lower than the ED₅₀ for antagonism of a second receptor (e.g., mGluR1).

Examples of Group I mGluR antagonists include, but are not limited to, any of the following (E)-6-methyl-2-styryl-pyridine (SIB 1893), 6-methyl-2-(phenylazo)-3-pyridinol, methyl-4-carboxyphenylglycine (MCPG), or 2-methyl-6-(phenylethynyl)-pyridine (MPEP). Examples of Group I mGluR antagonists also include those described in, e.g., U.S. patent application Ser. Nos. 10/076,618; 10/211,523; and 10/766,948. Examples of mGluR5-selective antagonists include, but are not limited to those described in, e.g., U.S. Pat. No. 7,205,411 and U.S. patent application Ser. No. 11/523,873. Examples of mGluR1-selective antagonists include, but are not limited to, those described in, e.g., U.S. Pat. No. 6,482,824

In some embodiments, where a Group I mGluR antagonist (e.g., an mGluR5 antagonist) is administered in combination with a PAK inhibitor, the dose of the Group I mGluR antagonist ranges from about 0.001 mg/kg/day to about 7.0 mg/kg/day, e.g., about 0.005 mg/kg/day, 0.009 mg/kg/day, 0.010 mg/kg/day, 0.050 mg/kg/day, 0.20 mg/kg/day, 0.50 mg/kg/day, 0.75 mg/kg/day, 1.0 mg/kg/day, 2.0 mg/kg/day, 3.5 mg/kg/day, 4.5 mg/kg/day, 5.0 mg/kg/day, 6.2 mg/kg/day, 6.8 mg/kg/day, or any other dose from about 0.001 mg/kg/day to about 7.0 mg/kg/day.

In some embodiments, the combination treatment comprises administering a combined dosage form that is a pharmacological composition comprising a therapeutically effective amount of a PAK inhibitor and a Group I mGluR antagonist (e.g., an mGluR5-selective antagonist) as described herein. In some embodiments, the pharmacological composition comprises a PAK inhibitor compound and an mGluR5-selective antagonist selected from U.S. Pat. No. 7,205,411.

### EXAMPLES

The following specific examples are to be construed as merely illustrative, and not limitative of the remainder of the disclosure in any way whatsoever.

### Example 1

### Identification of Compounds Having High Affinity for PAK Active Sites

The present example describes the identification of small molecule compounds that have high affinity for the active site of one or more PAK isoforms. A competitive binding assay was utilized, which was developed by Ambit, Inc. (San Diego, Calif.), comprising three components: (1) an immobilized kinase “bait” probe (e.g., staurosporine) having high affinity for the catalytic site of multiple kinases; (2) full length PAK or a PAK catalytic domain expressed on the surface of T7 bacteriophage; and (3) a candidate PAK inhibitor substance (“test substance”) in solution in a series of known concentrations. When these three components are combined, the test substance is tested for its ability to compete, in a concentration-dependent manner, with the immobilized kinase bait probe for binding for binding to the phage-PAK catalytic domain. Afterwards, the amount of bait probe-bound phage-PAK is detected, for example, by a phage plaque assay and/or quantitative PCR of phage DNA. The amount of probe-bound phage-PAK is inversely proportional to the affinity of the candidate inhibitor for the kinase and is used in determining a Kd value of the test substance for the PAK catalytic site, as described below. The assay is described in further detail in Fabian et al. (2005, Nat. Biotech., 23:329) and Carter et al. (2005, Proc. Natl. Acad. Sci., USA, 102:11011); both of which are incorporated herein by reference.

### Materials and Methods

### Preparation of Kinase Fusion Constructs and of Phage Expressing Kinase Fusion Constructs

Various PAK isoforms and/or their catalytic domains were cloned in a modified version of the commercially available T7 Select 10-3strain (Novagen). The head portion of each phage particle included 415 copies of the major capsid protein, and kinase fusion proteins are in approximately one to ten of these. Fusion proteins are randomly distributed across the phage head surface. The N terminus of the kinase is fused to the C terminus of the capsid protein through a flexible peptide linker. Kinases are linked to the T7 phage particle but are not incorporated into the phage head. Fusion proteins are randomly incorporated, and therefore distributed across the phage head surface. Clones of each kinase were sequenced, compared to an appropriate reference sequence, changed by site-directed mutagenesis where necessary to exactly match the reference sequence throughout the kinase domain, and transferred into the phage vector.

### Kinase Assays

T7 kinase-tagged phage strains were grown in parallel in 24- or 96-well blocks in an E. coli host derived from the BL21 strain. E. coli were grown to log phase and infected with T7 phage from a frozen stock (multiplicity of infection ˜0.1) and incubated with shaking at 32° C. until lysis (approximately 90 minutes). Lysates were centrifuged (6,000×g) and filtered (0.2 inn) to remove cell debris. Streptavidin-coated magnetic beads were treated with staurosporine for 30 minutes at 25° C. to generate affinity resins for kinase assays. Liganded beads were blocked with excess biotin and washed with blocking buffer (SeaBlock [Pierce], 1% BSA, 0.05% Tween-20, 1 mM DTT) to remove unbound ligand and to reduce nonspecific phage binding. Binding reactions were assembled by combining phage lysates, liganded affinity beads and test compounds in 1× binding buffer (20% SeaBlock, 0.17×PBS, 0.05% Tween-20, 5 mM DTT). Test substances were prepared as 1,000× stocks in DMSO and rapidly diluted into the aqueous environment (0.1% DMSO final). DMSO (0.1%) was added to control assays lacking a test substance. All reactions were carried out in polystyrene 96-well plates that had been pretreated with blocking buffer in final volume of 0.1 ml. Assay plates were incubated at 25° C. with shaking for 1 hour, (e.g. long enough for binding reactions to reach equilibrium) and affinity beads were washed four times with wash buffer (1×PBS, 0.05% Tween-20, 1 mM DTT) to remove unbound phage. After the final wash, beads were resuspended in elution buffer (1×PBS, 0.05% Tween-20, 2 μM nonbiotinylated affinity ligand) and incubated at 25° C. with shaking for 30 minutes. Phage titer in the eluates was measured by standard plaque assays or by quantitative PCR.

Equilibrium binding equations yield the following expression for the binding constant for the interaction between the free test substance and the kinase (Kd (test)), assuming that the phage concentration is below Kd (test):

K_{d}(test)=(K_{d}(probe)/(K_{d}(probe)+[Probe])))*[test]½

IC_{d} (probe) is the binding constant for the interaction between the kinase and the immobilized ligand, [Probe] is the concentration of the immobilized ligand [test] ½ is the concentration of the free candidate compound at the midpoint of the transition. If [Probe] is below K_{d} (probe) the expression simplifies to K_{d} (test)=[test] ½. Under these conditions, binding constants measured for the interaction between kinases and candidate compounds (IC_{d} (test)) are therefore independent of the affinity of the immobilized ligand for the kinase (K_{d} (probe)). T7 phage grow to a titer of 108-1010 plaque forming units (PFU)/ml, and the concentration of phage-tagged kinase in the binding reaction is therefore in the low pM range. The concentration of the immobilized ligand is kept in the low nM range, below its binding constant for the kinase. Binding data were fit to the equation:

PFU=L+((H−L)*(K_{d}(test)/(K_{d}(test)+[candidate])))

where L is the lower baseline, H is the upper baseline, IC_{d} (test) is the binding constant for the interaction between the test substance and the kinase, and [test] is the free test substance concentration. Binding constants measured in duplicate on the same day as part of the same experiment generally were within twofold. Duplicate measurements performed on separate days generally varied by no more than fourfold. For kinase/test substance combinations where no interaction was observed, the binding constant was arbitrarily set to 1 M. K_{d} values were converted to p K_{d} (−log K_{d}), and clustering was based on the Pearson correlation. For further details of the assay system, see, for example, Fabian et al. (2005, Nat. Biotech., 23:329) and Carter et al. (2005, Proc. Natl. Acad. Sci., USA, 102:11011); both of which are incorporated herein by reference.

Using the above-described assay system, the interaction of a panel of 38 small molecule test substances was tested with the active sites of six PAK kinases: PAK1, PAK2, PAK3, PAK4, PAK6, and PAK7. As shown in Table 1, several compounds were found to have a K_{d} value less than 10 μM for one or more of the tested PAK isoforms.

TABLE 1
Results of Test PAK Inhibitor Affinity Assay

| | BMS- | | | | | | | | | | | | | | | | |
|---|---|---|---|---|---|---|---|---|---|---|---|---|---|---|---|---|---|
| | 387032/ | | CHI4-258/ | | | | JNJ- | | | | Stauro- | | | | | VX-680/ | |
| Kinase | SNS-032 | | TKI-258 | | EKB-569 | | 7706621 | | PKC-412 | | sporine | SU-14813 | | Sunitinib | | MK-0457 | |
| PAK1 | ε 10 | μM | ε 10 | μM | <5 | μM | ε 10 | μM | <5 | μM | <1 μM | ε 10 | μM | ε 10 | μM | <5 | μM |
| PAK2 | ε 10 | μM | ε 10 | μM | ε 10 | μM | ε 10 | μM | <5 | μM | <1 μM | ε 10 | μM | ε 10 | μM | <10 | μM |
| PAK3 | <5 | μM | <1 | μM | ε 10 | μM | <5 | μM | <1 | μM | <1 μM | <1 | μM | <0.2 | μM | <5 | μM |
| PAK4 | ε 10 | μM | ε 10 | μM | ε 10 | μM | <5 | μM | ε 10 | μM | <1 μM | ε 10 | μM | <5 | μM | <5 | μM |
| PAK6 | ε 10 | μM | ε 10 | μM | ε 10 | μM | <5 | μM | ε 10 | μM | <1 μM | ε 10 | μM | <5 | μM | ε 10 | μM |
| PAK7 | ε 10 | μM | ε 10 | μM | ε 10 | μM | <1 | μM | ε 10 | μM | <1 μM | ε 10 | μM | <1 | μM | <5 | μM |

Based on these data, specific compounds have been identified that have relatively high affinity for the catalytic domain of at least one PAK isoform, and are therefore useful inhibitors, as described herein. In some embodiments, such PAK inhibitors are used for the treatment and/or prophylaxis of mental disorders, e.g., Fragile X syndrome and autism spectrum disorders in animal models (e.g., FMR1KO mice), as described herein, and in human clinical trials, as described herein.

### Example 2

### Slice Electrophysiology Assay for Determination of PAK Inhibitory Activity

Materials: coronal cortical slices (400 μm) containing temporal cortex from 2- to 3-month-old C57-Black-6 mice male littermates (from Elevage Janvier, FRANCE) are prepared and allowed to recover in oxygenated (95% O2 and 5% CO2) warm (30° C.) artificial cerebrospinal fluid (ACSF) containing 124 mM NaCl, 5 mM KCl, 1.25 mM, NaH₂PO₄, 1 mM MgCl₂, 2 mM CaCl₂, 26 mM NaHCO₃, and 10 mM dextrose.

Compound dilution: a 10 mM DMSO stock solution is prepared for each test compound and 100 μL aliquots are stored at −20° C. On the day of experiment an aliquot is thawed and vortexed for fresh solutions preparation. The final concentration of DMSO is adjusted to 0.1% in all solutions, including control ACSF solution.

Perfusion: Artificial Cerebro-Spinal Fluid (ACSF) is perfused at 3 mL/min. The recording chamber has a volume of 1 mL. Then the chamber medium is renewed every 20 s. The perfusion liquid is maintained at 30±0.1° C.

Data acquisition: evoked-responses are sampled at 5 kHz before recording on the harddisk of the computer

Recording in cortical layer II/III: The recording is carried out on a Multi Electrode Array. Responses (field portentials) in layer II/III are evoked by layer IV stimulation between one MEA electrode and the GND electrode. I/O curve is first performed to define evoked responses for stimulation intensities between 100 and 800 μA, by 100 μA steps. The stimulus consists in a monopolar biphasic current pulse (negative for 60 μs and then positive for 60 μs) which is applied every 30 s to evoke “responses” (field Excitatory Post Synaptic Potentials; (fEPSP) in cortical layer II/III.

Basal synaptic transmission: a monopolar stimulation (a bi-phasic stimulus: ±300 mA for 120 ins between one MEA electrode and the GND) is applied every 30 s on the MPP fibres to evoke “responses” (field potentials: fEPSP) in the DG region. The basal stimulation intensity will be set to evoke 40% of maximal amplitude response. The same stimulation intensity will be used in the 100 Hz stimulation protocol.

LTP: a stimulus is applied every 30 s with an intensity settled at 40% of the maximal amplitude responses. LTP is then induced by TBS, which consists of eight brief bursts (each with four pulses at 100 Hz) of stimuli delivered every 200 ms. Potentiation of synaptic transmission is then monitored for an additional 40 minutes period. Since fEPSP result from glutamatergic synaptic transmission consecutive to afferent pathway stimulation, 10 μM NBQX are perfused on the slice, at the end of each experiment, to validate the glutamatergic nature of synaptic transmission as well as to subtract background noise at individual electrode level.

Compound evaluation: following a 10 minutes control recording period (to verify baseline stability), the compound is perfused for 20 minutes. Then, LTP is triggered and the fEPSP amplitude will be recorded for an additional 40 minutes period in the presence of compound.

Data analysis: fEPSP amplitudes are measured as the difference between the baseline (before stimulation) and the maximal peak amplitude. The fEPSP are normalized as a percent of the meanaveraged amplitude recorded over a 10 min control period, before compound application. Normalized fEPSP values are then averaged for each experiment carried out in control conditions and with the test compound. The fEPSP mean values (+/−SEM) are expressed as a function of time before and after LTP induction.

An increased LTP, compared to the baseline (Control), in a theta-burst stimulation protocol in the cortex indicates an increase in synaptic plasticity mediated by inhibition of PAK (FIGS. 1-3).

### Example 3

### Treatment of Schizophrenia by Administration of a PAK Inhibitor in an Animal Model

The ability of the PAK3-selective inhibitor SU-14813 to ameliorate behavioral and anatomical symptoms of schizophrenia (i.e., their mouse analogs) is tested in a dominant-negative DISC1 mouse model of schizophrenia (Hikida et al (2007), Proc Natl Acad Sci USA, 104(36):14501-14506). The structure of SU-14813 is as follows:

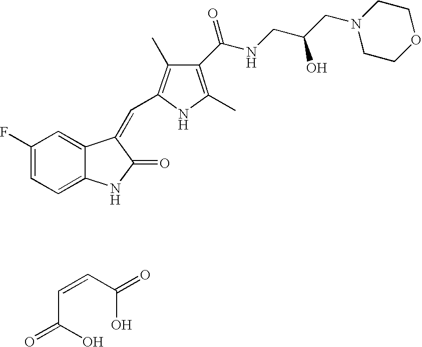

### 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide maleate

Forty DISC1 mice (ages 5-8 months) on a C57BL6 strain background are divided into a SU-14813 treatment group (1 mg/kg oral gavage) and a placebo group (0.1% DMSO in physiological saline solution) and analyzed for behavioral differences in open field, prepulse inhibition, and hidden food behavioral tests, with an interval of about one week between each type of test. In the open field test, each mouse is placed in a novel open field box (40 cm×40 cm; San Diego Instruments, San Diego, Calif.) for two hours. Horizontal and vertical locomotor activities in the periphery as well as the center area are automatically recorded by an infrared activity monitor (San Diego Instruments). Single breaks are reported as “counts.” In this behavioral test, a significant reduction in total activity in the SU-14813 group relative to the placebo group indicates a possible treatment effect.

In the hidden food test, mice are food-deprived for 24 h. After habituation to a new cage for 5 min, a food pellet is hidden under the cage bedding. The time it takes for the mouse to find the food pellet is measured until a maximum of 10 min is reached. In this behavioral test, a significant reduction in time to find the food pellet in the SU-14813 group relative to the placebo group is indicative of a successful treatment effect.

In the prepulse inhibition test, acoustic startle and prepulse inhibition responses are measured in a startle chamber (San Diego Instruments). Each mouse is subjected to six sets of seven trail types distributed pseudorandomly: pulse-alone trials, prepulse-pulse trials, and no-stimulus trials. The pulse used is 120 dB and the prepulse is 74 dB. A significant increase in the prepulse inhibition response in the SU-14813 group relative to the placebo group is indicative of a successful treatment effect.

In the forced swim test, each mouse is put in a large plastic cylinder, which is half-filled with room temperature water. The test duration is 6 min, during which the swim/immobility times are recorded. In this behavioral test, a significant reduction in immobility in the SU-14813 group relative to the placebo group is indicative of a successful treatment effect.

In order to evaluate the ability of SU-14813 to alter brain morphology, an MRI study is conducted on placebo-treated and SU-14813-treated groups of DISC 1-DN mice. In vivo MRI experiments are performed on an 11.7T Bruker Biospec small animal imaging system. A three-dimensional, fast-spin echo, diffusion weighted (DW) imaging sequence with twin navigation echoes is used to assess the ratio of lateral ventricle volume to total brain volume. A decrease in this ratio in the SU-14813-treated group relative to the ratio observed in the placebo-group is indicative of a successful treatment effect.

Statistical Analysis. Statistical analysis is performed by ANOVA or repeated ANOVA Differences between groups are considered significant at p<0.05.

### Example 4

### Treatment of Clinical Depression by Administration of a PAK Inhibitor in an Animal Model

A rat olfactory bulbectomy (OBX) model of clinical depression (see, e.g., van Riezen et al (1990), Pharmacal Ther, 47(1):21-34; and Jarosik et al (2007), Exp Neurol, 204(1):20-28) is used to evaluate treatment of clinical depression with a PAK inhibitor compound TKI-258 (4-amino-5-fluoro-3-[6-(4-methyl-1-piperazinyl)-1H-benzimidazol-2-yl]-2(1H)-quinolinone]; M.W. 392.4). Dendritic spine density and morphology are compared in treated and untreated groups of animals as described below. It is expected that treatment of OBX animals with TKI-258 will cause an increase in spine density relative to that observed in untreated OBX animals.

All experiments are performed in strict accordance with NIH standards for laboratory animal use. The study uses 48 adult male Sprague-Dawley rats (230-280 g) housed in groups of four animals (two sham and two OBX), as indicated in van Riezen et al supra, in a controlled environment with food and water available ad libitum. Half of the experimental animals (n=24) undergo bilateral olfactory bulbectomy (OBX) while the other half undergo sham surgery (n=24). Upon completion of surgery, animals are allowed to recover for 2 weeks prior to behavioral testing. This is necessary to: 1) allow for the recovery of animal body weight which is reduced following surgery, 2) allow complete healing of superficial surgical sites, and) “bulbectomy syndrome” develops during the first 2 weeks postsurgery.

Two weeks after surgery, OBX and sham-operated animals are subdivided into one of four experimental conditions. One group of OBX animals is administered daily injections of saline solution (n=6 for each surgical condition) or TKI-258 (1 mg/kg; oral gavage) (n=6 for each surgical condition). These groups are included to examine the effect of chronic administration of a PAK inhibitor (TKI-258) on olfactory bulbectomized animals (2 weeks postsurgical recovery+2 weeks TKI-258 treatment). Administration of the drug or control solution are given at the same time each day and in the home cage of each animal. Groups of OBX and sham-operated animals receive no treatment during this 2-week period and serve as unhandled controls. These groups are necessary to examine the persistence of observed effects of OBX on dendritic spine density (4 weeks postsurgery). Animals receiving postsurgery drug treatment are sacrificed 24 h after the last injection.

Animals are perfused transcardially with 4% formaldehyde (in 0.1 M sodium phosphate buffer, pH=7.4) under deep anesthesia with sodium pentobarbital (60 mg/kg) at the completion of experimental procedures. Following fixation, brains are removed and placed in 4% formaldehyde (freshly depolymerized from para-formaldehyde) overnight. Brains are then sectioned at 100 μm on a vibratome and prepared for Golgi impregnation using a protocol adapted from previously described methods (Izzo et al, 1987). In brief, tissue sections are postfixed in 1% OsO4 for 30 min and then washed in 0.1 M phosphate buffer (3×15 min). Sections are free-floated in 3.5% K2Cr2O7 solution for 90 min, mounted between two microscope slides in a “sandwich” assembly, and rapidly immersed in a 1% AgNO3 solution. The following day, sections are rinsed in ddH 2O, dehydrated in 70% and 100% ethanol, cleared with Histoclear™, and mounted on microscope slides with DPX.

Dendritic spines are counted on 1250× camera lucida images that include all spines observable in each focal plane occupied by the dendrite. Cells are analyzed only if they are fully impregnated (CA1: primary apical dendrites extended into stratum lacunosum moleculare and basilar dendrites extended into stratum oriens; CA3: primary apical dendrites extended into stratum lacunosum moleculare and basilar dendrites extended into stratum oriens; dentate gyms: secondary dendrites extended from primary dendrite within the molecular layer), intact, and occurring in regions of the section that are free of blood vessels, precipitate, and/or other imperfections. Dendritic spines are counted along the entire length of secondary oblique dendritic processes (50-100 μm) extending from the primary apical dendrite within stratum radiatum of area CA1 and CA3. In CA1 and CA3, secondary dendrites are defined as those branches projecting directly from the primary apical dendrite exclusive of tertiary daughter branches. In addition, spines are counted along the length of secondary dendrites of granule cells in the dentate gyms to determine if effects are limited to CA1 and CA3. In dentate gyrus, secondary dendrites are analyzed in the glutamatergic entorhinal input zone in the outer two-thirds of the molecular layer. Approximately 20 dendritic segments (10 in each cerebral hemisphere; 50-100 μm in length) in each hippocampal subregion (CA1, CA3, and dentate gyms) are examined for each experimental animal. Representative Golgi impregnated neurons from each hippocampal subfield are illustrated in FIG. 1. Treatment conditions are coded throughout the entire process of cell identification, spine counting, dendritic length analysis, and subsequent data analysis. Analysis of variance and Tukey post-hoc pairwise comparisons are used to assess differences between experimental groups.

When significant changes in dendrite spine density are observed, camera lucida images and the Zeiss CLSM measurement program are used to quantify the number and length of secondary dendrites. This analysis is necessary as apparent changes in dendritic spine density can result from an increase or decrease in the length of dendrites and not the formation or loss of spines per se. Photomicrographs are obtained with a helium-neon 633 laser and Zeiss 410 confocal laser scanning microscope.

### Example 5

### Treatment of Epilepsy by Administration of a PAK Inhibitor in an Animal Model

A rat tetanus toxin model of epilepsy is used to evaluate treatment of epilepsy with a PAK autoinhibitory domain (PAD) polypeptide expressed using an AAV expression vector (AAV-PAD). The sequence of the PAD sequence is:

HTIHVGFDAVTGEFTGMPEQWARLLQTSNITKSEQKKNPQAVLDVLEFYN
SKKTSNSQKYMSFTDKS

Details of AAV vector construction are described in, e.g., U.S. Pat. No. 7,244,423.

Wistar rat pups (Harlan Sprague Dawley, Indianapolis, Ind.), 10 d of age, are anesthetized with an intraperitoneal injection of ketamine and xylazine (33 and 1.5 mg/kg, respectively). When necessary, this is supplemented by inhalation of methoxyflurane (Metofane). Tetanus toxin solution to be injected is generated by dissolving 2.5 or 5 ng of tetanus toxin in 20 or 40 nl of sterile saline solution. Afterwards, the tetanus toxin solution is coinjected into the right hippocampus along with 10⁸ particles of AAV-PAD.

To inject tetanus toxin and the AAV-PAD, the pups are placed in an infant rat stereotaxic head holder, a midline incision is made, and a small hole is drilled in the skull. The stereotaxic coordinates for injection are: anteroposterior, −2.1 mm; mediolateral, 3.0 mm from the bregma; and dorsoventral, −2.95 mm from the dural surface. The toxin and AAV particles are slowly injected at 4 nl/min. After injection, the needle is left in place for 15 min to reduce reflux up the needle track During injections, the body temperature of rat pups is maintained by a warmed (electrically regulated) metal plate. Littermates, stereotaxically injected with sterile saline, or untreated rats serve as controls.

The frequency of behavioral seizures is monitored for 1 hr/day for 10 consecutive days after tetanus toxin/AAV injections. The types and duration of seizures are scored. Wild running seizures are most easily identified.

After seizure scoring on the 10th day animals are perfused transcardially and dendritic spines in the CA3 region are counted and analyzed as described in Example 2.

The t test for comparison of two independent means is used in comparing the number of seizures in treated vs untreated rats and in comparing dendritic and axon arbors in experimental and control rats. When data are not normally distributed, a Mann-Whitney U test is used. Sigma Stat is used to perform all statistical tests. It is expected that treatment with the AAV-PAD will reduce the frequency and severity of seizures.

### Example 6

### In Vivo Monitoring of Dendritic Spine Plasticity in Double Transgenic GFP-M/DN-DISC1 Mice Treated with a PAK Inhibitor

In the following experiment, dendritic spine plasticity is directly monitored in vivo by two photon laser scanning microscopy (TPLSM) in double transgenic GFP-M/DN-DISC1 mice treated with a PAK inhibitor (SU14813) or a placebo. Mice (C57BL/6) expressing GFP in a subset of cortical layer 5 neurons (transgenic line GFP-M described in Feng at al, 2000, Neuron 28:41-51) are crossed with DN-DISC1 C57BL/6 DN-DISC1 mice (Hikida et al (2007), Proc Natl Acad Sci USA, 104(36):14501-14506) to obtain heterozygous transgenic mice, which are then crossed to obtain homozygous double transgenic GFPM/DN-DISC1 mice used in this study.

GFP-M/DN-DISC1 animals aged 28-61 d are anesthetized using avertin (16 μl/g body weight; Sigma, St. Louis, Mo.). The skull is exposed, scrubbed, and cleaned with ethanol. Primary visual, somatosensory, auditory, and motor cortices are identified based on stereotaxic coordinates, and their location is confirmed with tracer injections (see below).

Long-term imaging experiments are started at P40. The skull is thinned over the imaging area as described in Grutzendler at al, (2002), Nature, 420:812-816. A small metal bar is affixed to the skull. The metal bar is then screwed into a plate that connected directly to the microscope stage for stability during imaging. The metal bar also allows for maintaining head angle and position during different imaging sessions. At the end of the imaging session, animals are sutured and returned to their cage. Thirty animals previously imaged at P40 are then divided into a control group receiving a 1% sugar solution (oral gavage once per day) and a treatment group administered SU14813, a PAK inhibitor, in 0.1% DMSO (oral gavage. 1 mg/kg, once per day). During the subsequent imaging sessions (at P45, P50, P55, or P70), animals are reanesthetized and the skull is rethinned. The same imaging area is identified based on the blood vessel pattern and gross dendritic pattern, which generally remains stable over this time period.

At the end of the last imaging session, injections of cholera toxin subunit B coupled to Alexa Fluor 594 are made adjacent to imaged areas to facilitate identification of imaged cells and cortical areas after fixation. Mice are transcardially perfused and fixed with paraformaldehyde, and coronal sections are cut to verify the location of imaged cells. Sections are then mounted in buffer, coverslipped, and sealed. Images are collected using a Fluoview confocal microscope (Olympus Optical, Melville, N.Y.).

For in vivo two photon imaging, a two-photon laser scanning microscope is used as described in Majewska et al, (2000), Pflügers Arch, 441:398-408. The microscope consists of a modified Fluoview confocal scan head (Olympus Optical) and a titanium/sulphur laser providing 100 fs pulses at 80 MHz at a wavelength of 920 nm (Tsunami; Spectra-Physics, Menlo Park, Calif.) pumped by a 10 W solid-state source (Millenia; Spectra-Physics). Fluorescence is detected using photomultiplier tubes (HC125-02; Hamamatsu, Shizouka, Japan) in whole-field detection mode. The craniotomy over the visual cortex is initially identified under whole-field fluorescence illumination, and areas with superficial dendrites are identified using a 20×, 0.95 numerical aperture lens (IR2; Olympus Optical). Spiny dendrites are further identified under digital zoom (7-10×) using two-photon imaging, and spines 50-200 μm below the pial surface are studied. Image acquisition is accomplished using Fluoview software. For motility measurements, Z stacks taken 0.5-1 μm apart are acquired every 5 min for 2 h. For synapse turnover experiments, Z stacks of dendrites and axons are acquired at P40 and then again at P50 or P70. Dendrites and axons located in layers 1-3 are studied. Although both layer 5 and layer 6 neurons are labeled in the mice used in this study, only layer 5 neurons send a clear apical dendrite close to the pial surface thus, the data will come from spines on the apical tuft of layer 5 neurons and axons in superficial cortical layers.

Images are exported to Matlab (MathWorks, Natick, Mass.) in which they are processed using custom-written algorithms for image enhancement and alignment of the time series. For motility measurements (see Majewska et al, (2003), Proc Natl Acad Sci USA, 100:16024-16029) spines are analyzed on two-dimensional projections containing between 5 and 30 individual images; therefore, movements in the z dimension are not analyzed. Spine motility is defined as the average change in length per unit time (micrometers per minute). Lengths are measured from the base of the protrusion to its tip. The position of spines are compared on different imaging days. Spines that are farther than 0.5 μm laterally from their previous location are considered to be different spines. Values for stable spines are defined as the percentage of the original spine population present on the second day of imaging. Only areas that show high signal-to-noise ratio in all imaging sessions will be considered for analysis. Analysis is performed blind with respect to animal age and sensory cortical area. Spine motility (e.g., spine turnover), morphology, and density are then compared between control and treatment groups. It is expected that treatment with a PAK inhibitor SU14813 will rescue defective spine morphology relative to that observed in untreated control animals.

### Example 7

### Clinical Trial: Treatment of Schizophrenia with a PAK Inhibitor

The following human clinical trial is performed to determine the safety and efficacy of a PAK inhibitor EKB-569 for the treatment of schizophrenia. EKB-569 has the following structure:

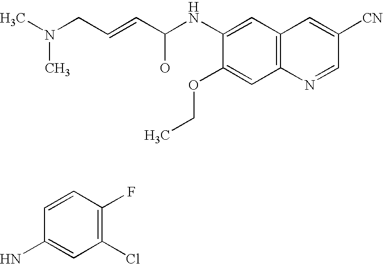

Sixty patients are recruited via referrals from community mental health teams, after the patients have been diagnosed with schizophrenia using the Structured Clinical Interview for DSM-IV (“SCID”; First et al., (1995), Structured Clinical Interview for DSM-IV Axis I Disorders, Patient Edition (SCID-P), version 2, New York State Psychiatric Institute, Biometrics Research, New York).

A screening visit is arranged and a full explanation of the study prior to screening is provided if the patient appeared suitable for and interested in taking part. For inclusion, all patients are required to meet the following criteria: (i) aged between 18 and 60 years, (ii) receiving stable treatment with an atypical (Risperidone, Olanzapine, Quetiapine) antipsychotic and have stable psychotic symptoms (i.e. no change in medication/dose of current medication over last 6 weeks and unlikely to require change in antipsychotic medication), (iii) negative urine screening for illicit drugs and negative pregnancy test for female patients, (iv) cooperative, able to ingest oral medication and willing to undertake repeated cognitive testing, (v) able to provide written informed consent, (vi) reading ability of not more than 40 errors on the National Adult Reading (Nelson et al, (1991)), and (vii) between 1 and 2 standard deviations (S.D.) below expected performance on the basis of age and education level on the California Verbal Learning Test (Delis et al., 1987). In addition, the following criteria are used to define unsuitable patients: (i) concurrent DSM-IV diagnosis, (ii) current treatment with benzodiazepines or antidepressants, (iii) history of neurodegenerative disorder in first degree relative (e.g. AD, Parkinson's disease, Huntington's disease, multiple sclerosis), (iv) history of DSM-IV substance dependence in the last year or substance abuse within last month, (v) lifetime history of trauma resulting in loss of consciousness for 1 h or longer, (vi) participation in another investigational drug trial within 6 weeks prior to study entry, (vii) recent (within last 3 months) history of suicidal or violent acts, and (viii) current diagnosis of uncontrollable seizure disorder, active peptic ulceration, severe and unstable cardiovascular disease or/and acute severe unstable asthma. The study procedures are approved by an institutional ethics review board. All patients in the study must provide written informed consent.

After screening has identified suitable patients that have provided informed consent, patients are placed on a single-blind placebo for 1 week. After 1 week on placebo (baseline), all patients complete a comprehensive cognitive test battery and undergo clinical assessments, and then are randomized into a double-blind protocol so that, half of the sample received EKB-569 capsules and the remaining half received placebo for the next 24 weeks. Cognitive and clinical assessments are carried out again at 12 weeks and 24 weeks.

Patients assigned to the EKB-569 group will receive 1.5 mg twice a day for the first 2 weeks, 3 mg twice a day over the next 2 weeks, 4.5 mg twice a day dose for the next 2 weeks and then 6 mg twice a day for the remaining period so at the time of 12 weeks cognitive assessments all patients are on the maximum dose. The placebo group will receive identical appearing capsules containing ascorbic acid (100 mg).

Symptoms are rated within 4 days of cognitive testing using the Positive and Negative Syndrome scale (PANSS) (Kay et al. (1987), Schizophr Res, 13:261-276) on all three occasions. Side effects are also assessed within 4 days of testing using the Abnormal Involuntary Movement Scale (AIMS) (Guy, (1976), ECCDEU Assessment Manual for Psychopharmacology (revised), DHEW Publication No. (ADM) National Institutes of Mental Health, Rockville, Md., pages 76-338). Inter-rater reliability is carried out for PANSS at 6 monthly intervals by rating exemplar cases based on patient interviews on videotapes.

The cognitive battery includes measures of executive functioning, verbal skills, verbal and spatial working memory, attention and psychomotor speed. The battery is administered to all patients on all three occasions in the same fixed order. Patients are allowed to take breaks as needed in order to obtain maximal performance at all times. Tests are administered and scored by trained psychologists who are blind to patients' group affiliations and are not involved in patients' treatment plan in any way.

Patients are told that the aim of the study is to investigate the cognitive effects of EKB-569. They are requested to abstain from alcohol for at least 24 h prior to their scheduled cognitive testing.

The patients in the EKB-569 and placebo groups are compared on demographic, clinical, and cognitive variables obtained at baseline using independent sample I-tests.

The effects of EKB-569 on positive symptoms, negative symptoms, general psychopathology score, total PANSS scores, and the scores on the AIMS are analyzed (separately) by 2 (Treatment: EKB-569, placebo)×3 (Time: baseline, 12 weeks, 24 weeks) analysis of variance (ANOVA).

All cognitive variables are first examined for their distribution properties, i.e., to ensure normality. The cognitive effects of EKB-569 over time are then evaluated by Treatment×Time ANOVA, performed separately for each variable, with Time as a within-subjects factor and Treatment as a between-subjects factor, followed by post-hoc mean comparisons wherever appropriate. All cognitive effects are then re-evaluated using ANOVA performed separately on change scores computed for each variable (12 weeks data minus baseline data, 24 weeks data minus baseline data). Alpha level for testing significance of effects is p=0.05.

The disclosed embodiments are provided by way of example only. Numerous variations, changes, and substitutions are feasible. It should be understood that various alternatives to the embodiments of the methods and compositions described herein may be employed in practicing the invention. It is intended that the following claims define the scope of the invention and that methods and compositions within the scope of these claims and their equivalents be covered thereby.

1-2. (canceled)

3. A method for reversing some or all defects in dendritic spine morphology, dendritic spine size, dendritic spine density and/or spine plasticity in a subject with a neuropsychiatric condition comprising administering to the subject at least once an effective amount of at least one of the compounds selected from the group consisting of: a Group I PAK transcription inhibitor, a Group I PAK translation inhibitor, a Group I PAK clearance agent, an agent that binds a Group I PAK to prevent its interaction with one or more cellular proteins, a Group I PAK autoinhibitory domain polypeptide or a fragment thereof and a Group I PAK antagonist.

4. The method of claim 1, wherein the neuropsychiatric condition is Huntington's disease.

5. The method of claim 1, wherein the compound is a Group I PAK transcription inhibitor.

6. The method of claim 1, wherein the compound is a Group I PAK translation inhibitor.

7. The method of claim 1, wherein the compound is an antisense nucleic acid molecule.

8. The method of claim 1, wherein the compound is an RNAi.

9. The method of claim 1, wherein the compound is a silencing RNA.

10. The method of claim 1, wherein the compound is a Group I PAK clearance agent.

11. The method of claim 1, wherein the compound is an agent that binds Group I PAK to prevent its interaction with one or more cellular proteins.

12. The method of claim 1, wherein the compound is a Group I PAK antagonist.

13-27. (canceled)

28. The method of claim 1, wherein the compound is a Group I PAK autoinhibitory domain polypeptide or a fragment thereof.

29. The method of claim 1, wherein the neuropsychiatric condition is schizophrenia.

30. The method of claim 1, wherein the neuropsychiatric condition is age-related cognitive decline.

31. The method of claim 1, wherein the neuropsychiatric condition is clinical depression.

32. The method of claim 1, wherein the defect in dendritic spine size is a reduction in dendritic spine surface area.

33. The method of claim 1, wherein the defect in dendritic spine size is a reduction in dendritic spine volume.

34. The method of claim 1, wherein the defect in dendritic spine density is a reduction of dendritic spine density.
